(19) United States
(12) Patent Application Publication
(10) Pub. No.: US 2002/0006397 A1
(43) Pub. Date: Jan. 17, 2002

(54) In VIVO loading of MHC

(76) Inventors: Bruce L. Roberts, Southboro, MA (US); Srinivas Shankara, Shrewsbury, MA (US)

Correspondence Address: GENZYME CORPORATION, LEGAL DEPARTMENT, 15 PLEASANT ST CONNECTOR, FRAMINGHAM, MA, 01701-9322, US

(21) Appl. No.: 09/843,342

(22) Filed: Apr. 25, 2001

Related U.S. Application Data
(60) Provisional application No. 60/200,562, filed on Apr. 28, 2000.

(51) Int. Cl. A61K 48/00; C07H 21/04; A61K 39/255; C12N 9/00; A61K 39/27; C12N 5/08

(52) U.S. Cl. 424/93.21; 424/229.1; 435/372.3; 435/183; 536/23.2

(57) ABSTRACT

The present invention provides several embodiments that ultimately result in the in vivo loading of endogenous antigenic peptides from a target cell. The invention also presents a method for inducing an immune response to an endogenous antigen in a subject by delivering an effective amount of an agent that stimulates in vivo loading of the endogenous antigen into an Antigenic Peptide Binding Protein (“APBP”). The APBP presents the endogenous antigen to a T cell in vivo. A polynucleotide encoding an APBP is delivered to a target cell under conditions such that the APBP is expressed in the target cell. Endogenous antigenic peptides bind the APBP forming an APBP:peptide complex. A cytotoxic agent also is administered to the subject and delivered to the target cell in an amount effective to lyse the target cell which releases the complexes. The complexes present the antigenic peptide to a T cell or an antigen presenting cell (APC) which mounts the immune response. An effective amount of an antigen presenting cell (APC) recruitment factor can be administered to the subject to recruit APC to the locus of the target cell. APCs take up the APBP:peptide complexes and the peptides are processed and presented by MHC molecules to T cells in vivo.

### TECHNICAL FIELD

This invention is in the field of molecular immunology and medicine. In particular, methods of inducing an antigen-specific immune response against endogenous antigens are provided.

### BACKGROUND

The mammalian immune system is capable of generating responses to foreign antigens, to self antigens present on pathological cells, and to self antigens present on normal tissues. The immune system comprises two types of antigen-specific cells: B cells and T cells. B cells synthesize both membrane-bound and secreted antibody. T cells can be characterized phenotypically by the manner in which they recognize antigen, by their cell surface markers, and by their secreted products. T cells express distinctive membrane molecules. Included among these are the T cell antigen receptor (TCR), which appears on the cell surface in association with CD3; and accessory molecules such as CD5, CD28 and CD45R. Subpopulations of T cells can be distinguished by the presence of additional membrane molecules. Thus, for example, T cells that express CD4 recognize antigen associated with class II MHC molecules and generally function as helper cells whose roles include enhancement of antibody production by B cells, while T cells that express CD8 recognize antigen associated with class I MHC molecules and generally function as cytotoxic cells.

Immune cells recognize discrete sites, known as epitopes or antigenic determinants, on the antigen. Epitopes are regions of an immunogen or antigen that bind to antigen-specific membrane-bound receptors on immune cells or to their soluble counterparts, such as antibodies. Both membrane-bound antibody on the surface of a B lymphocyte and secreted antibody recognize soluble antigen. Unlike B cells, which recognize soluble antigen, T cells recognize antigen only when the antigen is associated with self major histocompatibility complex (MHC) gene products on the surface of an antigen presenting cell. This antigen can be displayed together with MHC molecules on the surface of antigen-presenting cells, on virus-infected cells, pathological cells such as cancer cells, and grafts.

Disease states can result from invasion by pathogenic organisms, including bacterial, viral, and protozoan pathogens, and subsequent inefficient or ineffective immune response to the invader. Disease states can also result from various causes such as misfunctioning of cellular physiology or breakdown in cellular control mechanisms that regulate cell growth and division. In such cases it would be useful to be able to induce the natural host immune system to recognize the diseased cells and mount a specific immune response to control or eliminate the pathological tissue. However, the specific mechanism governing the function of the immune system together with molecular processes mediated by pathological cells frequently limit the ability of the immune system to react to diseased cells. A particularly well-studied case is cancer.

Although most tumor cells express on their surface specific antigens that are foreign to the host body, the host immune system often fails to develop an effective immune protection against the residing tumor cells. In other words, the surface proteins may be tumor specific and antigenic, yet the residing tumor cells lack strong immunogenicity. While the exact reasons for such immunological unresponsiveness are not entirely clear, several molecular and cellular factors have been suggested as critical for eliciting an effective, systemic antigen-specific CTL, including, for example, the immunogenicity of particular tumor antigens; the abundance of the MHC-antigen complex presentation for T-cell recognition; the presence and activity of co-stimulatory factors associated with APCs; and the function of cytokines.

A number of tumor antigens recognized by CTL have been identified, most of which are melanoma-related. (See Lindauer et al. (1998) J. Mol. Med. 76:32-47, for a comprehensive list of melanoma-related and melanoma-unrelated tumor antigens). The melanoma-related tumor antigens so far identified result from either differentiation antigens which are normal self proteins overexpressed by melanocyte (gp100, MART-1), proteins selectively expressed in tumor cells but not in most normal cells (MAGE family), or mutated gene products (CDK4, MUM-1). Non-melanoma related tumor antigens so far identified result from either a mutant HLA-A2 molecule, overexpressed HER-2/neu and p53 proteins, viral antigens (HPV 16 E6 and E7), or MUC-1 which is not MHC-restricted. Attempts to identify differentiation antigens overexpressed by non-melanoma cancers such as breast, lung, prostate, ovarian or colon carcinomas that might be good candidates for CTL have largely been unsuccessful. Moreover, vaccines using the so far identified non-melanoma antigens have largely been weak in immunogenicity or short lived. The results, most of the cancer immunotherapy trials conducted to date have been for the treatment of melanoma and little is available to offer patients suffering with other malignant diseases. Because it is difficult to identify the precise antigens expressed by the complete range of tumor types and the properties of these tumors vary from subject to subject, it would be also useful to have methods which are capable of stimulating immune responses to antigens expressed by a subject's specific tumor cells. Methods to induce an immune response to such endogenous antigens would also be applicable to other pathological conditions such as intracellular infections and cellular pathologies. These methods would be especially useful if they could function in vivo as well as ex vivo.

Finally, the processing and presentation of peptide antigens as well as the affinity of these presented antigens for antigen-recognizing T cell receptors depends on the precise three dimensional configuration of the peptide antigen as it is complexed with the antigen presenting molecule. A select sub-set of all possible peptide sequences is particularly effective at inducing immune responses against cells that express the corresponding peptide containing protein. In stimulating an immune response against pathological cells it is valuable to enhance the recognition by the host immune system of such particularly immunogenic peptide sequences as a means of stimulating cytotoxic attack directed at the target cells. For this purpose, it is useful to select particularly antigenic peptide sequences as components of a broad-based immunotherapy. In addition, the precise configuration of the antigen presenting MHC molecules is essential to effective antigen presentation. Manipulation of MHC molecule configuration has the potential to further enhance antigen presentation and recognition.

To address the complexity of modulating the diverse components of the immune system and the difficulty of mounting effective immunotherapy against pathological cells in vivo, the following invention provides a series of complementary compositions and methods for inducing immune responses to endogenous antigens in a subject.

### DISCLOSURE OF THE INVENTION

The present invention provides several embodiments that ultimately result in the in vivo loading of endogenous antigenic peptides from a target cell. When the target cell is a cancer cell, e.g, a breast cancer cell, a prostate cancer cell, a head and neck cancer cell or a lymphoma cell, the method is useful to treat or inhibit the growth of tumors and progression of the disease. Thus, this invention also provides a method to inhibit the proliferation of a cancer cell by priming the immune system of a subject having the tumor.

One of skill in the art can determine if the object of the method has been met by noting reduction in tumor burden or tumor mass, or alternatively, an amelioration of symptoms normally associated with the presence of a particular tumor or cancer.

In one embodiment, the invention is a method of inducing an immune response to an endogenous antigen in a subject by delivering an effective amount of an agent that stimulates in vivo loading of the endogenous antigen into an Antigenic Peptide Binding Protein (“APBP”). The APBP presents the endogenous antigen to a T cell in vivo.

A polynucleotide encoding an APBP is delivered to a target cell under conditions such that the APBP is expressed in the target cell. Endogenous antigenic peptides bind the APBP forming an APBP:peptide complex. A cytotoxic agent also is administered to the subject and delivered to the target cell in an amount effective to lyse the target cell which releases the complexes. The complexes present the antigenic peptide to a T cell or an antigen presenting cell (APC) which mounts the immune response. An effective amount of an antigen presenting cell (APC) recruitment factor can be administered to the subject to recruit APC to the locus of the target cell. APCs take up the APBP:peptide complexes and the peptides are processed and presented by MHC molecules to T cells in vivo.

Because one purpose of the cytotoxic agent is to release antigen-specific APBP:peptide complexes from a target cell at the time when these complexes are maximally concentrated, it is desirable to control when the cytotoxic agent will destroy the cell. In one embodiment, this is achieved by controlling the temporal expression of the cytotoxic agent and peptide binding protein genes or by delivering the cytotoxic compound (or gene encoding the compound) to the target cells at a time after the delivery of the APBP. Alternatively, a conditionally active cytotoxic gene that encodes a protein that is cytotoxic in itself when supplemented with a cofactor or activating component or that is able to generate a cytotoxic compound when provided with an appropriate cofactor or prodrug may be administered and activated at the appropriate time.

In one embodiment, the polynucleotides encoding the cytotoxic agent and the APBP are delivered in separate gene delivery vehicles. Here, it is unlikely that all the target cells that express one polynucleotide will express the other. Therefore, to maximize destruction of those cells containing high intracellular levels of APBP:peptide complexes, it is desirable for the cytotoxic agent to have a significant bystander effect, i.e. lyse cells neighboring the cells into which the gene delivery vehicle was successfully introduced and transcribed. The need for a cytotoxic agent having a significant bystander effect is greatly reduced in the embodiment where the polynucleotides encoding the cytotoxic agent and the APBP are delivered in the same gene delivery vehicle.

In a further embodiment of the invention, an antigen-specific immune response is induced in a subject by delivering an effective amount of an APBP, a cytotoxic agent and an effective amount of antigen presenting cell (APC) recruitment factor. In this aspect, the APBP, cytotoxic agent and APC recruitment factor can be delivered as whole molecules; via polynucleotides encoding these molecules; or as combinations of whole molecules and polynucleotides. Suitable APC recruitment factors are discussed herein.

APBP include, but are not limited to heat shock proteins and MHC molecules selected from the group consisting of a soluble MHC class I molecule, an antigen presenting matrix, a multimer of soluble MHC class I molecules and an antibody engineered to bind antigenic peptides is administered to the subject in an amount effective to deliver the MHC to the target cell to bind the endogenous antigenic peptides so that they bind the antigenic peptides and present them to a T cell in vivo. In an alternative aspect of the invention, the agent that stimulates in vivo loading of an APBP is an agent that stimulates in vivo fusion of a target cell containing endogenous antigens with a macrophage.

In a yet further aspect of the invention the agent that stimulates in vivo loading of an APBP is a polynucleotide encoding a rejection antigen and the antigen is administered to the subject. In this embodiment, an immune response to the rejection antigen is induced prior to, subsequently to or concurrently with administration of the rejection antigen so as to educate cytotoxic T cells against the rejection antigen. When the rejection antigen is expressed on the surface of the target cell, the cytotoxic T cells lyse the cell and release endogenous antigens. The endogenous antigens bind APBPs to form APBP:peptide complexes. In one aspect, the APBP:peptide complexes directly present the antigenic peptide to a T cell which in turn, induces the immune response against the endogenous antigen and lyses cells expressing the endogenous antigens. In an alternative embodiment, the APBP:peptide complexes present the antigenic peptide to an antigen presenting cell, e.g., a dendritic cell.

In an alternative embodiment, target cells are genetically altered in vivo to force them to produce cell surface proteins that can be recognized by either unmodified APCs or genetically altered APCs. Recognition of these “marked” tumor cells by APCs leads to the engulfing of tumor cells by the APCs by any of a number of different means including phagocytosis or to the fusion of tumor cells with DCs via cell membrane fusion. Engulfing or fusion of the “marked” tumor cells by/with APCs effectively causes the tumor antigens and respective antigen genes of the tumor cell to be transferred inside the APC where they are available for entry into antigen processing pathways and presentation on the surface of the APC in the content of co-stimulatory signals thus favoring the generation of an anti-tumor cell immune response.

The union of APCs with tumor cells can be promoted by a variety of means including 1) taking advantage of the inherent ability of DCs to engulf microbes and cells via Fc receptors, mannose receptors and receptors for surface markers of apoptotic cells, 2) taking advantage of the natural tropism of viral envelope proteins such as HIV gp120 for DC cell surface molecules such as CD4, 3) causing natural or novel ligands or receptors to be displayed on the surface of tumor cells which have affinity for naturally occurring ligands or receptors displayed on the surface of dendritic cells or 4) introducing novel ligands into tumor cells and the cognate receptors into DCs or vice versa. In all of these cases, genetic modification of tumor cells enhances the likelihood that the “marked” tumor cells will be engulfed by APCs and cancer cell derived antigens or their corresponding genes will be delivered into the APCs.

This invention also provides an in vivo method of producing a multimer of soluble MHC class I molecules and an antibody to bind antigenic peptides. The invention further provides an in vivo method to produce a fusion polypeptide comprising a T cell antigen presenting domain fused to an oligomerization domain. In one aspect, the T cell antigen presenting domain comprises a plurality of immunoglobulin fold domains of an MHC class I molecule.

The subject invention also provides a method for inducing lysis of a target cell in a subject by inducing an immune response to an exogenous rejection antigen in the subject and delivering to the target cell an effective amount of a polynucleotide encoding the exogenous antigen.

The methods described above may be further modified by delivering to the subject an effective amount of autologous and/or allogeneic APC and/or T cells co-administered to the subject. In further embodiment, the APC and/or T cells are genetically modified to express an effective amount of a cytokine and/or a co-stimulatory factor. In a yet further aspect of this invention, an MHC selected from the group consisting of a soluble MHC class I molecule, an antigen presenting matrix, a multimer of soluble MHC class I molecules and an antibody engineered to bind antigenic peptides is administered to the subject in an amount effective to deliver the MHC to the target cell to bind the endogenous antigenic peptides so that they bind the antigenic peptides and present them to a T cell in vivo. As is apparent to those of skill in the art, the methods of these inventions can be combined with well known and/or yet to be discovered therapeutic methods and compositions.

The preferred aspect of this invention requires in vivo practice of the invention. However, the methods described herein can be practiced in vivo, ex vivo or in vitro. In particular, the APBP, the cytotoxic agent and the optional APC recruiting factor can be delivered directly to a subject as molecules or encoded for by polynucleotides in gene delivery vehicles. Alternatively, ex vivo methods involve subjecting cells obtained from the subject to the methods described herein and administering those cells back to the patient. Finally, the methods of the present invention can be used in an in vitro cell culture to generate antigen-specific immune effector cells. These cells can then be used as a therapeutic agent.

### MODES FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

General Techniques

The practice of the present invention employs, unless otherwise indicated, conventional techniques of molecular biology (including recombinant techniques), microbiology, cell biology, biochemistry and immunology, which are within the skill of the art. Such techniques are explained fully in the literature, such as, MOLECULAR CLONING: A LABORATORY MANUAL, SECOND EDITION (Sambrook et al., 1989); CURRENT PROTOCOLS IN MOLECULAR BIOLOGY (F. M. Ausubel et al., eds., 1987); OLIGONUCLEOTIDE SYNTHESIS (M. J. Gait, ed., 1984); ANIMAL CELL CULTURE (R. I. Freshney, ed., 1987); METHODS IN ENZYMOLOGY (Academic Press, Inc.); HANDBOOK OF EXPERIMENTAL IMMUNOLOGY (D. M. Wei & C. C. Blackwell, eds.); GENE TRANSFER VECTORS FOR MAMMALIAN CELLS (J. M. Miller & M. P. Calos, eds., 1987); PCR: THE POLYMERASE CHAIN REACTION, (Mullis et al., eds., 1994); CURRENT PROTOCOLS IN IMMUNOLOGY (J. E. Coligan et al., eds., 1991); ANTIBODIES: A LABORATORY MANUAL (E. Harlow and D. Lane eds. (1988)); PCR 2: A PRACTICAL APPROACH (M. J. MacPherson, B. D. Hames and G. R. Taylor eds. (1995)); and ANIMAL CELL CULTURE (R. I. Freshney, ed. (1987)).

Definitions

As used herein, certain terms may have the following defined meanings.

As used in the specification and claims, the singular form “a”, “an” and “the” include plural references unless the context clearly dictates otherwise. For example, the term “a cell” includes a plurality of cells, including mixtures thereof.

The term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The term “ antigen” is well understood in the art and includes substances which are immunogenic, i.e., immunogens, as well as substances which induce immunological unresponsiveness, or anergy, i.e., anergens.

A “native” or “natural” antigen is a polypeptide, protein or a fragment which contains an epitope, which has been isolated from a natural biological source, and which can specifically bind to an antigen receptor, in particular a T cell antigen receptor (TCR), in a subject.

A “self-antigen” also referred to herein as a native or wild-type antigen is an antigenic peptide that induces little or no immune response in the subject due to self-tolerance to the antigen. An example of a self-antigen is the human melanoma antigen gp100.

An “altered antigen” is one having a primary sequence that is different from that of the corresponding wild-type antigen. Altered antigens can be made by synthetic or recombinant methods and include, but are not limited to, antigenic peptides that are differentially modified during or after translation, e.g., by phosphorylation, glycosylation, cross-linking, acylation, proteolytic cleavage, linkage to an antibody molecule, membrane molecule or other ligand. (Ferguson et al. (1988) Ann. Rev. Biochem. 57:285-320). A synthetic or altered antigen of the invention is intended to bind to the same TCR as the natural epitope.

“Endogenous antigens” means components of normal and pathological cells in a subject and include cellular constituents derived from intracellular infectious agents such as viruses, bacteria and intracellular parasites. Endogenous antigens are intended to include any cellular component, e.g., proteins, lipoproteins, glycoproteins, lipids, carbohydrates, nucleic acids, steroids, prostaglandins, and combinations and complexes thereof.

The term “tumor associated antigen” or “TAA” refers to an antigen that is associated with or specific to a tumor. Examples of known TAAs include gp100, MART and MAGE.

“Cellular constituents” are intended to include any cellular component, e.g., proteins, lipoproteins, glycoproteins, lipids, carbohydrates, nucleic acids, steroids, prostaglandins, and combinations and complexes thereof.

The term “lysing” refers to the action of rupturing the cell wall and/or cell membrane to release cellular constituents. These constituents include cytoplasmic and cell surface components in soluble or insoluble form.

By “priming” is meant any treatment or preparation causing a desired result.

An “antigenic peptide binding protein” is any protein which is capable of binding peptides, preferably peptides that elicit an antigenic response. For example, heat shock proteins, antigen-presenting matrix proteins and major histocompatibility complex (MHC) molecules each have a binding groove that enables them to bind to peptide sequences.

The terms “major histocompatibility complex” or “MHC” refers to a complex of genes encoding cell-surface molecules that are required for antigen presentation to T cells and for rapid graft rejection. In humans, the MHC complex is also known as the HLA complex. The proteins encoded by the MHC complex are known as “MHC molecules” and are classified into class I and class II MHC molecules. Class I MHC molecules include membrane heterodimeric proteins made up of an □ chain encoded in the MHC associated noncovalently with □2-microglobulin. Class I MHC molecules are expressed by nearly all nucleated cells and have been shown to function in antigen presentation to CD8+ T cells. Class I molecules include HLA-A, -B, and -C in humans. Class II MHC molecules also include membrane heterodimeric proteins consisting of noncovalently associated A and B chains. Class II MHC are known to function in CD4⁺ T cells and, in humans, include HLA-DP, -DQ, and DR.

The term “heat shock protein (HSP)” refers the class of proteins present in most animals which are encoded for by genes that are transcribed suddenly, quickly, and with coordination when the animal is exposed to certain types of stress such as a sudden temperature increase. Non-limiting examples of HSP are HSP gp96, HSP90, HSP70, HSP65, HSP28 and the like. These are commercially available from StressGen Biotechnologies, Victoria, Canada.

The term “antigen-presenting matrix”, as used herein, intends a molecule or molecules that present antigen in such a way that the antigen can be bound by a T cell antigen receptor on the surface of a T cell. An antigen-presenting matrix can be on the surface of an antigen-presenting cell (APC), on a vesicle preparation of an APC, or can be in the form of a synthetic matrix on a solid support such as a bead or a plate. An example of a synthetic antigen-presenting matrix is purified MHC class I molecules complexed to β2-microglobulin, multimers of such purified MHC class I molecules, purified MHC Class II molecules, or functional portions thereof, attached to a solid support.

A “cytotoxic agent” includes proteins and other molecules that are toxic to a cell, for example by disrupting the cell's normal function, and can induce cell lysis. Cytotoxic agents include molecules that are inherently toxic to a cell and molecules that are “conditionally activated”. A conditionally activated agent generates a cytotoxin when provided with an “activating compound”. A prodrug is an example of a conditionally activated cytotoxic agent.

The terms “antigen presenting cell recruitment factors” or “APC recruitment factors” includes both intact, whole cells as well as other molecules capable of recruiting antigen presenting cells. Examples include molecules such as interleukin 4 (IL-4), granulocyte macrophage colony stimulating factor (GM-CSF), Sepragel and macrophage inflammatory protein 3 alpha (MIP3α), which are commercially available from Genzyme (Framingham Mass.), Immunex, Schering-Plough and R&D Systems (Minneapolis, Minn.). They can be recombinantly produced using the methods disclosed in F. M. Ausubel et al., (1987), supra. Peptides, proteins and compounds having the same biological activity as the above-noted factors are included within the scope of this invention.

For purposes of the present invention, it is contemplated that a single peptide or protein could possess two or more of the three functions described above, i.e. antigen presenting cell recruitment, cytotoxic agents and/or antigenic peptide binding protein. This multifunctional molecule may be encoded for by a naturally occurring gene or, alternatively, may be engineered using recombinant techniques.

The term “allogeneic” as used herein, indicates the origin of a cell. Thus, a cell being administered to individual (the “recipient”) is allogeneic if the cell was derived from an individual not genetically identical to the recipient; in particular, the term relates to non-identity in expressed MHC molecules. An allogeneic cell can also be a progeny of an allogeneic cell. The term also indicates that cells of different cell types are derived from genetically non-identical donors, or if they are progeny of cells derived from genetically non-identical donors. For example, an APC is said to be allogeneic to an effector cell if they are derived from genetically non-identical donors.

The term “immune effector cells” refers to cells capable of binding an antigen and which mediate an immune response. These cells include, but are not limited to, T cells, B cells, monocytes, macrophages, NK cells and cytotoxic T lymphocytes (CTLs), for example CTL lines, CTL clones, and CTLs from tumor, inflammatory, or other infiltrates. Certain diseased tissue expresses specific antigens and CTLs specific for these antigens have been identified. For example, approximately 80% of melanomas express the antigen known as gp100.

The term “immune effector molecule” as used herein, refers to molecules capable of antigen-specific binding, and includes antibodies, T cell antigen receptors, and MHC Class I and Class II molecules.

A “naive” immune effector cell is an immune effector cell that has never been exposed to an antigen.

As used herein, the term “educated, antigen-specific immune effector cell”, is an immune effector cell as defined above, which has encountered antigen and which is specific for that antigen. An educated, antigen-specific immune effector cell may be activated upon binding antigen. “Activated” implies that the cell is no longer in Go phase, and begins to produce cytokines characteristic of the cell type. For example, activated CD4⁺ T cells secrete IL-2 and have a higher number of high affinity IL-2 receptors on their cell surfaces relative to resting CD4⁺ T cells.

An antigenic peptide or polypeptide of the invention may be preferentially recognized by antigen-specific immune effector cells, such as B cells and T cells. In the context of T cells, the term “recognized” intends that a peptide or polypeptide of the invention, comprising one or more antigenic epitopes, is recognized, i.e., is presented on the surface of an APC together with (i.e., bound to) an MHC molecule in such a way that a T cell antigen receptor (TCR) on the surface of an antigen-specific T cell binds to the epitope wherein such binding results in activation of the T cell. The term “preferentially recognized” intends that a polypeptide of the invention is substantially not recognized, as defined above, by a T cell specific for an unrelated antigen. Assays for determining whether an epitope is recognized by an antigen-specific T cell are known in the art and are described herein.

As used herein, the term “inducing an immune response in a subject” is a term well understood in the art and intends that an increase of at least about 2-fold, more preferably at least about 5-fold, more preferably at least about 10-fold, more preferably at least about 100-fold, even more preferably at least about 500-fold, even more preferably at least about 1000-fold or more in an immune response to an antigen (or epitope) can be detected or measured, after introducing the antigen (or epitope) into the subject, relative to the immune response (if any) before introduction of the antigen (or epitope) into the subject. An immune response to an antigen (or epitope), includes, but is not limited to, production of an antigen-specific (or epitope-specific) antibody and production of an immune cell expressing on its surface a molecule, which specifically binds to an antigen (or epitope).

The term “antigen presenting cells (APC)” refers to a class of cells capable of presenting one or more antigens in the form of antigen-MHC complex recognizable by specific effector cells of the immune system, and thereby inducing an effective cellular immune response against the antigen or antigens being presented. While many types of cells may be capable of presenting antigens on their cell surface for T-cell recognition, only professional APCs have the capacity to present antigens in an efficient amount and further to activate T-cells for cytotoxic T-lymphocyte (CTL) response. APCs can be intact whole cells such as macrophages, B-cells and dendritic cells; or other molecules, naturally occurring or synthetic, such as purified MHC class I molecules complexed to β2-microglobulin.

The term “dendritic cells (DC)” refers to a diverse population of morphologically similar cell types found in a variety of lymphoid and non-lymphoid tissues (Steinman (1991) Ann. Rev. Immunol. 9:271-296). Dendritic cells constitute the most potent and preferred APCs in the organism. A subset, if not all, of dendritic cells are derived from bone marrow progenitor cells, circulate in small numbers in the peripheral blood and appear either as immature Langerhans' cells or terminally differentiated mature cells. While the dendritic cells can be differentiated from monocytes, they possess distinct phenotypes. For example, a particular differentiating marker, CD14 antigen, is not found in dendritic cells but is possessed by monocytes. Also, dendritic cells are not phagocytic, whereas the monocytes are strongly phagocytosing cells. It has been shown that DCs provide all the signals necessary for T cell activation and proliferation. “Co-stimulatory molecules” are involved in the interaction between receptor-ligand pairs expressed on the surface of antigen presenting cells and T cells. Research accumulated over the past several years has demonstrated convincingly that resting T cells require at least two signals for induction of cytokine gene expression and proliferation (Schwartz R. H. (1990) Science 248:1349-1356 and Jenkins M. K. (1992) Immunol. Today 13:69-73). One signal, the one that confers specificity, can be produced by interaction of the TCR/CD3 complex with an appropriate MHC/peptide complex. The second signal is not antigen specific and is termed the “co-stimulatory” signal. This signal was originally defined as an activity provided by bone-marrow-derived accessory cells such as macrophages and dendritic cells, the so called “professional” APCs. Several molecules have been shown to enhance co-stimulatory activity. These are heat stable antigen (HSA) (Liu Y. et al. (1992) J. Exp. Med. 175:437-445), chondroitin sulfate-modified MHC invariant chain (Ii-CS) (Naujokas M. F. et al. (1993) Cell 74:257-268), intracellular adhesion molecule 1 (ICAM-1) (Van Seventer G. A. (1990) J. Immunol. 144:4579-4586), B7-1, and B7-2/B70 (Schwartz R. H. (1992) Cell 71:1065-1068). These molecules each appear to assist co-stimulation by interacting with their cognate ligands on the T cells. One exemplary receptor-ligand pair is the B7 co-stimulatory molecule on the surface of APCs and its counter-receptor CD28 or CTLA-4 on T cells (Freeman et al. (1993) Science 262:909-911; Young et al. (1992) J. Clin. Invest. 90: 229 and Nabavi et al. (1992) Nature 360:266-268). Other important co-stimulatory molecules are CD40, CD54, CD80, CD86. The term “co-stimulatory molecule” encompasses any single molecule or combination of molecules which, when acting together with a peptide/MHC complex bound by a TCR on the surface of a T cell, provides a co-stimulatory effect which achieves activation of the T cell that binds the peptide. The term thus encompasses B7, or other co-stimulatory molecule(s) on an antigen-presenting matrix such as an APC, fragments thereof (alone, complexed with another molecule(s), or as part of a fusion protein) which, together with peptide/MHC complex, binds to a cognate ligand and results in activation of the T cell when the TCR on the surface of the T cell specifically binds the peptide. Co-stimulatory molecules are commercially available from a variety of sources, including, for example, Beckman Coulter, Inc. (Fullerton, Calif.). It is intended, although not always explicitly stated, that molecules having similar biological activity as wild-type or purified co-stimulatory molecules (e.g., recombinantly produced or muteins thereof) are intended to be used within the spirit and scope of the invention.

As used herein, the term “cytokine” refers to any one of the numerous factors that exert a variety of effects on cells, for example, inducing growth or proliferation. Non-limiting examples of cytokines which may be used alone or in combination in the practice of the present invention include, interleukin-2 (IL-2), stem cell factor (SCF), interleukin 3 (IL-3), interleukin 6 (IL-6), interleukin 12 (IL-12), G-CSF, granulocyte macrophage-colony stimulating factor (GM-CSF), interleukin-1 alpha (IL-1), interleukin-11 (IL-11), MIP-11, leukemia inhibitory factor (LIF), c-kit ligand, thrombopoietin (TPO) and flt3 ligand. The present invention also includes culture conditions in which one or more cytokine is specifically excluded from the medium. Cytokines are commercially available from several vendors such as, for example, Genzyme (Framingham, Mass.), Genentech (South San Francisco, Calif.), Amgen (Thousand Oaks, Calif.), R&D Systems (Minneapolis, Minn.) and Immunex (Seattle, Wash.). It is intended, although not always explicitly stated, that molecules having similar biological activity as wild-type or purified cytokines (e.g., recombinantly produced or muteins thereof) are intended to be used within the spirit and scope of the invention.

A “gene” refers to a polynucleotide containing at least one open reading frame that is capable of encoding a particular polypeptide or protein after being transcribed and translated.

As used herein, the term “administration, administering, delivery and delivering” means providing to the subject the composition or agent to achieve the intended purpose of the method. When the agent is delivered to a subject such as a mouse, a rat or a human patient, the agent can be added to a pharmaceutically acceptable carrier and systemically or locally administered to the subject. Therapeutic amounts can be empirically determined and will vary with the pathology or condition being treated, the subject being treated and the efficacy and toxicity of the therapy.

The terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules.

The term “cDNAs” refers to complementary DNA, that is mRNA molecules present in a cell or organism made into cDNA with an enzyme such as reverse transcriptase. A “cDNA library” is a collection of all of the mRNA molecules present in a cell or organism; all turned into cDNA molecules with the enzyme reverse transcriptase, then inserted into “vectors”.

The term “peptide” is used in its broadest sense to refer to a compound of two or more subunit amino acids, amino acid analogs, or peptidomimetics. The subunits may be linked by peptide bonds. In another embodiment, the subunits are linked by other bonds, e.g. ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

A “fusion polypeptide” refers to a chimeric polypeptide molecule comprised of two or more polypeptides joined together by peptide bonds wherein the polypeptides do not naturally occur in a such a configuration.

A “domain” is a discrete portion of a polypeptide that is defined by its function. Examples include: a “T cell antigen presenting domain” which is a polypeptide that specifically binds an antigenic peptide. A “substrate-binding domain” which is any polypeptide domain which binds in a specific manner to a second molecule; a “secretion signal domain” which is a polypeptide that directs the secretion of a polypeptide molecule through the plasma membrane into the extracellular space; an “immunoglobulin fold domain” which refers to any of a family of related polypeptide domains which share elements of a conserved amino acid sequence common to this family and fold into a characteristic three dimensional immunoglobulin structure; and an “oligomerization domain” which is a multivalent domain that binds to itself or to a second multivalent platform molecule to form stable molecular multimers.

A platform molecule refers to a molecule that has multiple binding sites for peptide sequences of an oligomerization domain. Examples include but are not limited to strepavidin coated particles to bind peptide mimetics of biotin, heparin to bind heparin binding peptides, and receptor multimers to bind receptor specific ligands.

A “ligand” is a polypeptide which bind specifically to a receptor molecule. A “peptide mimetic” of a ligand is a polypeptide which binds specifically to the ligand binding site of a cell surface receptor although it is not the naturally occurring ligand for that receptor.

A “self-assembling protein” is a polypeptide which has the capacity to spontaneously form multimers with copies of itself or specific other molecules.

The term “genetically modified” means containing and/or expressing a foreign gene or nucleic acid sequence which in turn, modifies the genotype or phenotype of the cell or its progeny. In other words, it refers to any addition, deletion or disruption to a cell's endogenous nucleotides.

As used herein, “expression” refers to the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook et al. (1989) supra ). Similarly, an eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

“Under transcriptional control” is a term well understood in the art and indicates that transcription of a polynucleotide sequence, usually a DNA sequence, depends on its being operably (operatively) linked to an element which contributes to the initiation of, or promotes, transcription. “Operably linked” refers to a juxtaposition wherein the elements are in an arrangement allowing them to function.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “viral vector” is defined as a recombinantly produced virus or viral particle that comprises a polynucleotide to be delivered into a host cell, in vivo, ex vivo or in vitro. Examples of viral vectors include retroviral vectors, adenovirus vectors, adeno-associated virus vectors and the like.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. (see, e.g., WO 95/27071). Ads are easy to grow and do not require integration into the host cell genome. Recombinant Ad-derived vectors, particularly those that reduce the potential for recombination and generation of wild-type virus, have also been constructed. (see WO 95/00655 and WO 95/11984). Wild-type AAV has high infectivity and specificity integrating into the host cell's genome. Hernonat and Muzyczka (1984) Proc. Natl. Acad. Sci. 81:6466-6470 and Lebkowski et al. (1988) Mol. Cell. Biol. 8:3988-3996.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions of about 6×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

A polynucleotide or polynucleotide region (or a polypeptide or polypeptide region) that has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art, for example those described IN CURRENT PROTOCOLS IN MOLECULAR BIOLOGY (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/cgibin/BLAST.

“In vivo” gene delivery, gene transfer, gene therapy and the like as used herein, are terms referring to the introduction of a vector comprising an exogenous polynucleotide directly into the body of an organism, such as a human or non-human mammal, whereby the exogenous polynucleotide is introduced to a cell of such organism in vivo.

The term “isolated” means separated from constituents, cellular and otherwise, in which the polynucleotide, peptide, polypeptide, protein, antibody, or fragments thereof, are normally associated with in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eucaryotic cell in which it is produced in nature.

“Host cell”, “target cell” or “recipient cell” are intended to include any individual cell or cell culture which can be or have been recipients for vectors or the incorporation of exogenous nucleic acid molecules, polynucleotides and/or proteins. It also is intended to include progeny of a single cell, and the progeny may not necessarily be completely identical (in morphology or in genomic or total DNA complement) to the original parent cell due to natural, accidental, or deliberate mutation. The cells may be procaryotic or eucaryotic, and include but are not limited to bacterial cells, yeast cells, animal cells, and mammalian cells, e.g., murine, rat, simian or human.

A “subject” is a vertebrate, preferably a mammal, more preferably a human. Mammals include, but are not limited to, murines, simians, humans, farm animals, sport animals, and pets.

A “control” is an alternative subject or sample used in an experiment for comparison purpose. A control can be “positive” or “negative”. For example, where the purpose of the experiment is to determine a correlation of an altered expression level of a gene with a particular type of cancer, it is generally preferable to use a positive control (a subject or a sample from a subject, carrying such alteration and exhibiting syndromes characteristic of that disease), and a negative control (a subject or a sample from a subject lacking the altered expression and clinical syndrome of that disease).

The terms “cancer,” “neoplasm,” and “tumor,” used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition.

Tumor cell growth can be assessed by any means known in the art, including, but not limited to, measuring tumor size, determining whether tumor cells are proliferating using a ³H-thymidine incorporation assay, or counting tumor cells. “Suppressing” tumor growth means any or all of the following states: slowing, delaying, and “suppressing” tumor growth indicates a growth state that is curtailed when stopping tumor growth, as well as tumor shrinkage.

The term “culturing” refers to the in vitro propagation of cells or organisms on or in media of various kinds. It is understood that the descendants of a cell grown in culture may not be completely identical (morphologically, genetically, or phenotypically) to the parent cell. By “expanded” is meant any proliferation or division of cells.

An “agent” as used herein refers to a compound or composition that causes a change in a subject. Such agents include, but are not limited to chemical compounds, polynucleotides, polypeptides, lipids, polysaccharides and combinations and complexes thereof.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin REMINGTON'S PHARM. SCI., 15th Ed. (Mack Publ. Co., Easton (1975)).

An “effective amount” is an amount sufficient to effect beneficial or desired results. An effective amount can be delivered in one or more administrations, applications or dosages.

Embodiments of the Invention

In one aspect, the present invention provides a method for inducing an immune response to an endogenous antigen in a subject by delivering an agent that stimulates in vivo loading of the endogenous antigen into an Antigenic Peptide Binding Protein (APBP) molecule. The APBP molecule is loaded in vivo under conditions such that the endogenous antigen is presented in the context of the APBP molecule to a T cell. For example, an agent that targets a specific type of pathological cell is delivered to a patient suffering from a disease characterized by this type of cellular pathology. The agent contacts the target cells and induces the in vivo presentation of endogenous antigens from them to APBP which further present the endogenous antigens to T cells, thereby inducing an immune response against such pathologic cells.

In a further embodiment of the invention, the antigens are endogenous antigens of a tumor cell or a virally infected cell. Tumors suitably treated by this method include, but are not limited to malignant and benign tumors, such as hemangiomas, sarcomas, and carcinomas. Additional specific examples include, but are not limited to melanoma, head and neck cancer, breast cancer, lung cancer, and prostate cancer.

This method induces an immune response that leads to destruction of distant metastases following locoregional administration of the described molecules to an accessible target cell, such as a cutaneous, cancerous, skin lesion or nodule. The antigenic peptides within a cancer cell are loaded onto the abundant APBP and the APBP:peptide complexes are released from the cancer cell as the cell is destroyed by the action of the cytotoxic agent. The released APBP:peptide complexes are taken up by local APCs or those recruited to the treated site (e.g., by the APC recruitment factor) and this results in presentation of antigenic peptides. Once the peptides have been presented on the cells' surface, an anti-tumor cell immune response is mounted and these educated, antigen-specific immune effector cells destroy local and distant cells expressing the tumor-specific antigen.

Antigenic Peptide Binding Proteins

For purposes of the present invention, an antigenic peptide binding protein (APBP) includes any molecule, preferably a peptide that binds antigenic peptides. In a preferred embodiment, the APBP is of human origin. Perhaps the most well-known naturally occurring APBPs are the membrane-bound glycoproteins encoded for by the genes known as the major histocompatibility complex (MHC). These proteins are involved in presenting a wide variety of antigens on the surface of a cell. In the region of the protein farthest away from the cell membrane, a cleft (or groove) binds to an antigenic peptide created by proteolytic activity within the cell. Through a complex series of processing steps, the antigenic peptide bound to the groove in the MHC is then presented on the surface of the cell, where it can be recognized by the immune system.

In a particular embodiment of the invention, the APBP is a multimer of soluble MHC class I molecules and an antibody engineered to bind antigen peptides. In a further embodiment, the multimer is the in vivo expression product of a fusion polypeptide comprising a T cell antigen presenting domain fused to an oligomerization domain. This aspect of the invention is described in more detail below.

The term “APBP” also includes heat shock proteins (HSP), for example HSP65. Like the MHC proteins, the heat shock proteins have a peptide binding groove that bind short peptide sequences generated by intracellular protein proteolysis mediated by proteosome complexes. Heat shock proteins may also participate in the loading of these bound peptide fragments onto MHC molecules which are subsequently presented at the cell's surface. Thus, HSP:peptide complexes can provide a rich source of antigenic peptides, and complexes purified from a transformed cell can provoke immune responses specific to the transformed cell. When the APBP is an HSP, anti-HSP antibody assays can be used as a means to monitor the therapy using methods and commercially available materials from StressGen Biotechnologies, Victoria, Canada. Alternatively, using techniques well-known in the art and described in Harlow and Lane (1989) supra, anti-HSP monoclonal antibodies can be raised and used to monitor the therapy.

Cytotoxic Agents

The present invention also encompasses the use of a cytotoxic agent to lyse the target cell thereby releasing the APBP:peptide complex. Agents that are cytotoxic to cells include any substance that disrupts cellular function, preferably to a sufficient extent to lyse the target cell. In some aspects, the cytotoxic compound may exhibit a bystander effect, in that the compound is toxic to both the cells into which it is taken up and, in addition, to neighboring cells.

In one embodiment, the cytotoxic agent is cytotoxic without further modifications, e.g. toxins (e.g., ricin toxin), specific tumor suppressor gene products (e.g., p53), signaling molecules that can trigger cell death (e.g., Fas ligand and TNF-α), pro-apoptotic factors (e.g., bc1-xs as in Clarke, et al. (1995) PNAS 92:1104-11029), anti-sense RNA or ribozymes specific for mRNA encoding proteins vital for cell survival, proteases, DNAses and RNAses.

In a preferred embodiment, the cytotoxic agent is a prodrug that is activated by an activating agent. In other words, an activating compound must be administered to generate the cytotoxin. For example, herpes simplex virus thymidine kinase (HSV-tk) can be administered to a subject, either as a peptide or through a gene delivery vehicle carrying a polynucleotide encoding the peptide. However, HSV-tk does not become cytotoxic until an activating compound is added, for instance gancyclovir or acyclovir. (Available from Hoffman-LaRoche, Nutley, N.J.) Other examples of cytotoxic agents that do require a prodrug include E. coli cytosine deaminase, E. coli purine nucleoside phosphorylase, E. coli nitroreductase, mammalian cytochrome p450 isozymes, carboxypeptidase G2 and mammalian thymidine phosphorylase.

A Rejection Antigen as Cytotoxic Agent

In one embodiment, the present invention calls for the use of a rejection antigen that is utilized to treat a target cell such as a cancerous tissue where the rejection antigen is not expressed. A gene based vaccine encoding the rejection antigen (for instance a melanoma tumor antigen) is administered to a patient suffering from a malignancy other than melanoma so as to create a rejection antigen specific immune response. A gene based vector is then injected into the non-melanoma tumor so as to force the tumor cells to express the rejection antigen (in this case a melanoma tumor antigen) thereby rendering them susceptible to lysis by the rejection antigen specific immune effector cells that were generated in the patient by immunization with the vaccine encoding the rejection antigen. Lysis of the non-melanoma tumor mediated by rejection antigen specific T cells may lead to the release of endogenous antigens which may be captured and presented by APCs leading to a secondary anti-tumor cell immune response that could lead to the elimination of patient tumor cells that are not expressing the rejection antigen. Examples of rejection antigens include 1) any non-human protein that can provoke a potent cellular immune response in patients and 2) any human tumor antigen that can provoke a potent cellular immune response in patients and is not known to be expressed in a patient's tumor.

Any therapy that will provide rejection antigen-specific immune effector cells, e.g., cytotoxic T lymphocytes, that recognize and lyse any cell presenting the exogenous rejection antigen is intended to be within the scope of this invention. As is known to those of skill in the art, educated immune effector cells can be delivered to a subject or alternatively, antigen presenting cells engineered to present the rejection antigen in the context of an MHC molecule will educate naïve immune effector cells to educated, antigen-specific immune effector cells. In yet a further aspect, an antigenic epitope or gene based vaccine encoding the rejection antigen is delivered to the subject. The rejection antigen is presented by APC in vivo, which in turn, educate naive immune effector cells. These methods are discussed in more detail below.

Lysis of the target cell mediated by rejection antigen specific T cells leads to the release of endogenous antigenic cellular constituents. The target cell releases endogenous antigens which in turn, are internalized and processed by the subject's antigen presenting cells. Lysis of the target cell also leads to the release of cellular constituents including immunostimulatory factors and APC recruiting agents. The release of endogenous antigens, with the assistance of the immunostimulatory/attracting factors, mounts a secondary, immune response against all the target cells expressing the endogenous tumor antigens in the subject, resulting the eradication of the target cells.

Although any cell that comprises antigenic substances can be a target of the method of this invention, in one aspect, the target cell is a tumor cell and the exogeneous rejection antigen is any antigenic substance that can be introduced via gene therapy and presented by the tumor. An additional criteria is that the exogeneous rejection antigen can not be a “self-antigen” to the subject so that an immune response can be mounted by the patient against the rejection antigen. The exogenous rejection antigen is xenogeneic or allogeneic to the target tumor cells, i.e., it is not naturally present in the target tumor cells and “foreign” to the subject's immune system. Thus, the exogenous rejection antigen is in one aspect, a synthetic antigen that is capable of inducing an immune response in the subject. The term “antigen” is used in its broadest sense and includes minimal epitopes and chimeric molecules in addition to isolated full length proteins. As noted above, the antigen of this vaccine is homologous, heterologous (e.g., a murine antigen delivered to a human patient) or an altered antigen as compared to the corresponding native self-antigen. Antigens are of any type, e.g., tumor associated antigens or pathogenic antigens.

In one embodiment, the exogenous rejection antigen is a tumor associated antigen (“TAA”). TAAs include characterized tumor-associated antigens such as gp100 (Kawakami et al. (1997) Intern. Rev. Immunol. 14:173-192); MUC-1 (Henderson et al. (1996) Cancer Res. 56:3763-3770); MART-1 (Kawakami et al. (1994) Proc. Natl. Acad. Sci. 91:3515-3519; Kawakami et al. (1997) Intern. Rev. Immunol. 14:173-192; Ribas et al. (1997) Cancer Res. 57:2865-2869); HER-2/neu (U.S. Pat. No. 5,550,214); MAGE (PCT/US92/04354); HPV16, 18E6 and E7 (Ressing et al. (1996) Cancer Res. 56(1):582588; Restifo (1996) Current Opinion in Immunol. 8:658-663; Stern (1996) Adv. Cancer Res. 69:175-211; Tindle et al. (1995) Clin. Exp. Immunol. 101:265-271 and van Driel et al. (1996) Annals of Medicine 28:471-477); CEA (U.S. Pat. No. 5,274,087); PSA (Lundwall A. (1989) Biochem. Biophys. Research Communications 161:1151-59); prostate membrane specific antigen (PSMA) (Israeli et al. (1993) Cancer Research 53:227-30); tyrosinase (U.S. Pat. Nos. 5,530,096 and 4,898,814 and Brichard et al. (1993) J. Exp. Med. 178:489-49); tyrosinase related proteins 1 or 2 (TRP-1 and TRP-2); NY-ESO-1 (Chen et al. (1997) Proc. Natl. Acad. Sci. 94:1914-18); or the GA733 antigen (U.S. Pat. No. 5,185,254). The human and murine MUC1 coding sequences are provided under Genbank Accession No. M35093 and M64928.

It is known within the state of the art that minor modification to a nucleotide sequence will not affect the function of the molecules encoded thereby. Thus, biologically equivalent polynucleotides of published sequences are also useful in the methods described herein, as well as the polypeptides encoded thereby. Biologically equivalent polynucleotides can be identified by hybridization under stringent conditions (defined above) to the sequences disclosed in the published references or known in the art. Alternatively, the polynucleotides and polypeptides can be identified as being at least 80%, or more preferably, at least 90% or most preferably, at least 95%, identical (defined above) to the disclosed sequences using sequence alignment programs and default parameters. Polypeptides and polynucleotides having the above noted sequence identity to native antigen are intended to be encompassed within the term “altered antigen.”

In one aspect, multimers (concatemers) of an antigenic peptide, optionally including intervening amino acid sequences, is administered to the subject.

Also within the scope of this invention is the identification and use of an epitope or wild-type antigenic peptide corresponding to a yet unidentified protein. Methods are described below.

In an additional aspect of the invention, the methods include co-delivery of an effective amount of an antigen presenting cell recruitment factor. In yet a further aspect, an effective amount of a cytokine and/or co-stimulatory molecule is delivered to the subject.

The exogenous antigen can be introduced in the form of a polynucleotide coding for the antigen, alone or inserted into a gene delivery vehicle. It is understood by those of skill in the art that the polynucleotide is operatively linked to appropriate regulatory sequences for the transcription and translation of the polynucleotide into protein. In one aspect, the polynucleotide is delivered in a gene delivery vehicle as described above. In a further aspect, cytokines and/or co-stimulatory molecules are delivered in an amount to enhance the priming of the subject's immune system against the antigen. The cytokines and/or co-stimulatory molecules can be delivered as polynucleotides encoding the cytokines and/or co-stimulatory molecules. These can be delivered in the same or separate gene delivery vehicles as the polynucleotide encoding the antigen. Alternatively, compositions comprising the cytokines and/or co-stimulatory molecules can be delivered to the subject in an effective amount.

In an alternative aspect of the invention, the exogenous rejection antigen used to prime an initial immune response is introduced into the subject in the form of a cell-based vaccine. Cells used to deliver the antigen can be antigen-presenting cells including, but not limited to, dendritic cells (DCs), monocytes/macrophages, B lymphocytes or other cell types expressing the necessary immunostimulatory molecules as defined above. Methods for generating APCs expressing exogenous antigen are described below. These methods focus primarily on DCs, which are the more potent, therefore preferred APCs. DCs can be modified ex vivo or in vitro to express an effective amount of the exogenous rejection antigen. Modified DCs for use in the methods disclosed herein include, but are not limited to a foster antigen presenting cell, a dendritic cell hybrid, or a genetically modified DC. Methods for producing these DCs are known in the art and described below. Prior to administration the APCs are assayed to ensure that the exogenous rejection antigen is presented on the surface of the APC using methods known in the art and described below.

Alternatively, APCs expressing the exogenous rejection antigen of the invention are used in vitro to expand and isolate a population of immune effector cells which, in turn, are delivered to the subject as a form of adoptive immunotherapy. Prior to readministration in vivo, the educated immune effector cells are screened in vitro for their ability to lyse target cells expressing and presenting the exogenous antigen using methods known in the art, and described below.

Subsequent to the initial immunization with the rejection antigen, polynucleotides encoding the same exogenous rejection antigen are introduced directly to the target cells (such as cancer cells) in vivo to render them susceptible to the subject's primed immune system. In some embodiments of the invention, the antigen-encoding polypeptides are introduced by gene transfer technique, whereby a gene delivery vehicle is employed to deliver and express the transgene encoding the antigen. Various gene transfer techniques and gene delivery vehicles are known in the art and can be used to practice the present invention and are described herein. While the expression of the exogenous rejection antigen on target cells can be sufficient to induce lysis of the target tumor cells and subsequent immune response to endogenous tumor antigens that are released from the lysed tumor cells, it is desirable to further enhance this secondary immune response by, for example, providing immunostimulatory or attracting factors that can boost the presentation of the released endogenous tumor antigens and their recognition by host immune effector cells.

Identification, Isolation and Replication of Rejection Antigens

SAGE (U.S. Pat. No. 5,695,937) analysis can be employed to identify the antigens recognized by expanded immune effector cells such as CTLs. SAGE analysis involves identifying nucleotide sequences expressed in the antigen-expressing cells. Briefly, SAGE analysis begins with providing complementary deoxyribonucleic acid (cDNA) from (1) the antigen-expressing population and (2) cells not expressing that antigen. Both cDNAs can be linked to primer sites. Sequence tags are then created, for example, using the appropriate primers to amplify the DNA. By measuring the differences in these tags between the two cell types, sequences which are over-expressed in the antigen-expressing cell population can be identified.

Expression cloning methodology as described in Kawakami et al. (1994) Proc. Natl. Acad. Sci. 91:3515, also can be used to identify a novel tumor-associated antigen. Briefly, in this method, a library of cDNAs corresponding to mRNAs derived from tumor cells is cloned into an expression vector and introduced into target cells which are subsequently incubated with cytotoxic T cells. One identifies pools of cDNAs that are able to stimulate the CTL and through a process of sequential dilution and re-testing of less complex pools of cDNAs one is able to derive unique cDNA sequences that are able to stimulate the CTL and thus encode the cognate tumor antigen.

An antigen identification method, SPHERE, is described in PCT WO 97/35035. Briefly, an empirical screening method for the identification of MHC class I-restricted CTL epitopes is described that utilizes peptide libraries synthesized on a solid support (e.g., plastic beads) where each bead contains approximately 200 picomoles of a unique peptide that can be released in a controlled manner. The synthetic peptide library is tailored to a particular HLA restriction by fixing anchor residues that confer high-affinity binding to a particular HLA allele (e.g., HLA-A2) but contain a variable TCR epitope repertoire by randomizing the remaining positions. Roughly speaking, 50 96-well plates with 10,000 beads per well will accommodate a library with a complexity of approximately 5×10⁷. In order to minimize both the number of CTL cells required per screen and the amount of manual manipulations, the eluted peptides can be further pooled to yield wells with any desired complexity. Based on experiments with soluble libraries, it is to screen 10⁷ peptides in 96-well plates (10,000 peptides per well) with as few as 2×10⁶ CTL cells. After incubating with ⁵¹Cr-labeled APCs (e.g., T2 cells) and the CTL line(s), peptide pools containing reactive species can be determined by measuring ⁵¹Cr-release according to standard methods known in the art. Alternatively, cytokine production (e.g., interferon-γ) or proliferation (e.g., incorporation of ³H-thymidine) assays may be used. The sequence of the peptides on individual beads can be determined by sequencing residual peptide bound to the beads by, for example, N-terminal Edman degradation or other analytical techniques known to those of skill in the art.

Alternatively, muteins of the antigen as well as allogeneic antigens from a different species, of previously characterized antigens are useful in the subject invention. For example, MART-1 and gp100 are melanocyte differentiation antigens specifically recognized by HLA-A2 restricted tumor-infiltrating lymphocytes (TILs) derived from patients with melanoma, and appear to be involved in tumor regression (Kawakami Y. et al. (1994) Proc. Natl. Acad. Sci. 91:6458 and Kawakami Y. et al. (1994) Proc. Natl. Acad. Sci. 91:91:3515). Recently, the mouse homologue of human MART-1 has been isolated. The full-length open reading frame of the mouse MART-1 consists of 342 bp, encoding a protein of 113 amino acid residues with a predicted molecular weight of ˜13 kDa. Alignment of human and murine MART-1 amino acid sequences showed 68.6% identity.

The murine homologue of gp100 has also been identified. The open reading frame consists of 1,878 bp, predicting a protein of 626 amino acid residues which exhibits 75.5% identity to human gp100.

After isolation of the epitope or antigen, it can be expressed and purified using methods known in the art, including chromatography (e.g., ion exchange, affinity, and sizing column chromatography), centrifugation, differential solubility, or by any other standard technique for protein purification. For immunoaffinity chromatography, an epitope may be isolated by binding it to an affinity column comprising antibodies that were raised against that peptide, or a related peptide of the invention, and were affixed to a stationary support.

Alternatively, affinity tags such as hexa-His (Invitrogen), Maltose binding domain (New England Biolabs), influenza coat sequence (Kolodziej et al. (1991) Methods Enzymol. 194:508), and glutathione-S-transferase can be attached to the peptides of the invention to allow easy purification by passage over an appropriate affinity column. Isolated peptides can also be physically characterized using such techniques as proteolysis, nuclear magnetic resonance, and x-ray crystallography

In another alternative aspect, heterologous/altered antigens and peptides of the present invention can be synthesized using an appropriate solid state synthetic procedure. A preferred method is the Merrifield process. Merrifield (1967) Recent Progress in Hormone Res. 23:451. The antigenic activity of these peptides may conveniently be tested using, for example, the assays as described herein.

Also included within the scope of the invention are antigenic peptides that are differentially modified during or after translation, e.g., by phosphorylation, glycosylation, crosslinking, acylation, proteolytic cleavage, linkage to an antibody molecule, membrane molecule or other ligand, (Ferguson et al. (1988) Ann. Rev. Biochem. 57:285).

Antigen Presenting Cell (APC) Recruitment Factors

In one embodiment of the present invention, one or more factor(s) that recruit antigen presenting cells are also administered. The APC recruitment factor ensures that a high localized concentration of antigen presenting cells is available in the vicinity of the disrupted cancer cells to take up the liberated APBP:peptide complexes. The APCs recruited to the site have the ability to process the available APBP:peptide complexes in the context of MHC molecules, thereby inducing an anti-cancer immune response through education of immune effector cells.

Self-Assembling MHC Class I Multimers

In a specific aspect of the invention the APBP is a soluble multimer of an antigen binding MHC class I molecule. In particular, the present invention provides a novel efficient method for in vivo production of self-assembling multimers. A polynucleotide encoding an oligomeric domain is fused to the carboxy terminus of the antigen presenting domains of a MHC class I molecule. The polynucleotides are in turn, an aspect of a recombinant system comprising a polynucleotide encoding a fusion polypeptide together with a second polynucleotide that encodes a T cell epitope that binds specifically to the antigen presenting domain of the fusion polypeptide. In addition, the invention provides a host cell with a carefully selected genotype and a method of producing an antigen presenting fusion protein multimer, pre-loaded with T cell antigen. While prior methods have been described for preparing antigen presenting multimers loaded with T cell antigen peptides, they involved multiple steps and incorporated molecular and biochemical manipulations of both the antigen presenting molecule and the antigenic epitope which reduced the efficiency of the multimer preparation process while expanding the time and effort required to execute it. Thus, previous methods were time consuming and complex, requiring a high level of skill, and subject to variation in quality of the product produced from one preparation to the next. The present invention provides a significant improvement over previous methods because it eliminates the requirement for problematic biochemical and molecular modification of antigen presenting polypeptides by providing self-assembling multimers, and it also eliminates the need to prepare T cell antigen by a separate procedure thus increasing the efficiency of the key antigen loading process by producing antigen loaded molecules in a single step. Most significantly, the present invention can be practiced in vivo by delivery of the fusion protein encoding construct to tumor cells in a subject allowing for the direct presentation of tumor cell encoded antigens.

Peptide Compositions of Soluble MHC Class I Multimers

The present invention provides peptide compositions comprising fusion polypeptides further comprised of a T cell antigen presenting domain and an oligomerization domain. The T cell antigen presenting domain of the fusion polypeptide can be derived from any of a variety of naturally occurring T cell antigen presenting polypeptides, or it can be designed and synthesized by combining portions of various natural antigen presenting molecules with or without synthetic polypeptide segments. Those of skill in the art will recognize that specific MHC class I alleles can be selected to present antigens to a particular type of antigen specific T cell, a CD8⁺ cytotoxic T cell. These MHC class I polypeptides provide a number of T cell antigen presenting domains which are elements of the present invention.

Naturally occurring MHC class I antigen presenting molecules are comprised of a number of distinct polypeptide domains; an amino terminal secretion signal domain, three immunoglobulin fold domains, a transmembrane domain, and a cytoplasmic domain. Since it is well known that the antigen presenting function resides in the immunoglobulin fold domain, it is possible to construct soluble fusion proteins which perform the antigen presenting function by using just the immunoglobulin fold domains separated from the other naturally occurring domains of MHC class I molecules. Specific methods for producing engineered protein molecules are well known to those skilled in the art and are described in detail below.

In one embodiment of the invention the antigen presenting domain is comprised of a plurality of immunglobulin fold domains of an MHC class I molecule. In one aspect, these are the three immunoglobulin fold domains of an MHC class I molecule such as an HLA-A2 molecule, an alternative allele of the HLA-A gene, an allele of the HLA-B gene or an allele of the HLA-C gene.

The present invention provides for assembly of antigen presenting molecules into multimers by constructing a fusion polypeptide comprised of an antigen presenting domain fused to an oligomerization domain. Examples of oligomerization domains include but are not limited to various self-assembling polypeptides which form homomultimers such as a leucine zipper domain, ligands which bind to receptor molecules, peptide mimetics of ligands, and substrate binding domains such as a peptide mimetic of biotin.

The fusion polypeptides of the invention can form multimers spontaneously, without the need for further chemical modifications, by self assembly of the oligomerization domain or by binding of an oligomerization domain to a second platform molecule. For example, if the oligomerization domain is a self assembling molecule such as a leucine zipper, the fusion polypeptides will spontaneously form dimers in a solution with an appropriate salt concentration and pH. If the oligomerization domain is a polypeptide factor that form homotetramers, the fusion polypeptide will form tetramers. Alternatively, a fusion polypeptide with a ligand or substrate binding domain such as a peptide mimetic of biotin will form multimers in an appropriate buffer solution that contains a platform molecule covalently linked to a specific receptor molecule or oligomerization domain binding molecule. Thus, fusion polypeptides with an oligomerization domain comprising a peptide mimetic of biotin will form multimers when added to streptavidin coated beads. In a separate example, a substrate binding domain such as a heparin binding domain, will form multimers when added to a solution of heparin platform molecules. Alternatively, fusion polypeptides with a receptor specific ligand such as a growth factor will form multimers when added to a platform molecule such as a microbead coated with molecules of the polypeptide receptor for that growth factor. Or, a receptor coated platform such as a microbead will bind a peptide mimetic of the natural ligand for that receptor when the peptide mimetic of the ligand is fused to a T cell antigen presenting domain as an oligomerization domain.

Detailed information concerning the amino acids sequences of such polypeptides and of polynucleotides encoding these polypeptides can be obtained from various sequence databases such as GenBank, PIR and SWISSPROT. For example the human leucine zipper protein C/EBP gamma is described in GenBank accession number P53567 and the yeast leucine zipper protein GCN4 is described in GenBank accession number P03069. Additional information concerning the function and manipulation of such sequences is available in the scientific literature, see for example O'Shea et al. (1991) Science 254(5031):539-544; Agre et al. (1989) Science 246(4932):922-926; and Katz et al. (1989) Biotechniques 25(2):298-302. In addition, the amino acid sequence of a peptide mimetic of biotin (CHPQFC) has been determined by McLafferty et al. (1993) Gene 128:29-36. Many examples of ligands and receptors will be well known to practitioners of the art and specific sequences for such polypeptides and polynucleotides that encode them can be obtained from sequence databases such as listed here.

In a further aspect, the fusion polypeptides of the inventions comprise a secretion signal domain at their amino terminus that facilitates direction of the encoded polypeptide to the extracellular space. During this process, the cell removes the secretion signal to produce a fusion polypeptide as described above. The utility of this process for purification of the fusion polypeptide is described in detail below. A large number of specific secretion signal sequences have been identified and specific sequence information can be obtained from sequence databases. In addition, sequences have been assembled into protein domain databases such as SBASE and DOMO, where they can easily be accessed. Alternatively, the natural signal sequences occurring at the amino termini of MHC molecules can be employed.

Polynucleotides Encoding Peptides of the Invention

The present invention further comprises polynucleotides encoding peptides of the invention. Thus, the present invention provides polynucleotides encoding fusion polypeptides comprising an antigen presenting domain and an oligomerization domain. Such fusion polypeptides may also comprise a secretion signal domain. In addition, the present invention provides polynucleotides encoding T cell epitopes selected to bind specifically to a corresponding antigen presenting domain where the antigen presenting domain-T cell epitope complex specifically bind to a corresponding antigen specific T cell.

Techniques for constructing polynucleotides encoding selected polypeptide molecules such as fusion polypeptides and corresponding T cell peptide epitopes are well known to individuals skilled in the art. The nucleotide sequences of many antigen presenting molecules are known and cDNA clones of such genes have been produced. Similar nucleotide sequence data is available for many potential oligomerization domains and secretion signal domains. Thus one skilled in the art can design a polynucleotide which encodes a fusion protein of the invention by collecting the appropriate nucleotide sequences from a database such as GenBank and using a nucleotide sequence manipulation computer program such as is found in the Wisconsin GCG package, other commercially available software such as DNA Star, PG Gene, MacVector, or DNAsis, or by accessing sequence manipulation tools avalable on the world wide web such as at http://www.sdsc.edu/ResTools/cmshp.html, the CMS Molecular Biology Resource Page.

In designing a fusion polypeptide a practitioner of the art will join the sequences in an appropriate linear order and use a nucleic acid sequence manipulation program to translate the fused sequence to confirm that the amino acid encoding portions are aligned in the same open reading frame. One will also include a translation termination codon at the 3′ end of the fusion encoding open reading frame to end the polypeptide.

It is customary to design a recombinant DNA construction plan to facilitate assembly of the fusion encoding sequence. In some cases, it is possible to identify appropriately placed restriction endonuclease cleavage sites for cutting and splicing polypeptide encoding sequences in the desired way. Alternatively, it is common to design PCR primers to specifically amplify the sequences of interest. Such primers can be designed to incorporate convenient restriction sites for recombinant molecule assembly.

In constructing a recombinant polypeptide, the polypeptide encoding sequences to be included can be prepared from pre-existing cloned molecules, isolated from natural sources or synthesized by synthetic DNA chemistry, typically using an automated DNA oligonucleotide synthesizer.

When using pre-existing cloned sequences, fusion molecules can be assembled by cutting the sequences of interest with appropriate restriction endonucleases and joining the selected fragments by ligation. Alternatively, the appropriate sequences can be prepared by PCR using PCR primers designed to amplify the sequences of interest. The PCR fragments can then be cleaved with restriction enzymes or prepared for blunt end ligation and then joined by DNA ligase. Chosen polypeptide encoding sequences can also be prepared from natural sources. This may include genomic DNA but this is complicated by the fact that introns in the genomic sequence often disrupt the open reading frame. Thus it is common to prepare cDNA from an mRNA population isolated from a cell known to express the sequence of interest. This cDNA can then be used in an RT PCR reaction with PCR primers selected to amplify the sequence of interest for recombinant DNA manipulation.

In some cases it is desirable to synthesize oligonucleotides on an automated DNA synthesizer. This is especially true when the sequences of interest are relatively short such as less than 100 bases long. It is also possible to synthesize the entire fusion protein encoding polynucleotide and assemble the product using a series of overlapping oligonucleotides. As with DNA fragments prepared from clones or by PCR, the oligonucleotides are then joined by ligation.

While for some purposes it is possible to prepare and amplify polynucleotide sequences using PCR it is customary to clone the recombinant molecule by inserting it into an appropriate cloning vehicle, transforming the nucleic acid into a cell and propagating the recombinant cell. It is also customary to isolate recombinant polynucleotide from such a cell and analyze it by DNA sequence analysis to confirm that the construct that has the expected sequence.

The polynucleotides of this invention can be isolated using methods known in the art and described in the literature, e.g., replicated using PCR. The PCR technology is the subject matter of U.S. Pat. Nos. 4,683,195; 4,800,159; 4,754,065; and 4,683,202 and described in PCR: The Polymerase Chain Reaction (Mullis et al. eds, Birkhauser Press, Boston (1994)) or MacPherson, et al. (1991) and (1994), supra, and references cited therein. Alternatively, one of skill in the art can use the sequences provided herein and a commercial DNA synthesizer to replicate the DNA. Accordingly, this invention also provides a process for obtaining the polynucleotides of this invention by providing the linear sequence of the polynucleotide, nucleotides, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the polynucleotide into a suitable replication vector and insert the vector into a suitable host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

RNA can be obtained by first inserting a DNA polynucleotide into a suitable host cell. The DNA can be inserted by any appropriate method, e.g., by the use of an appropriate gene delivery vehicle (e.g., liposome, plasmid or vector) or by electroporation. When the cell replicates and the DNA is transcribed into RNA; the RNA can then be isolated using methods well known to those of skill in the art, for example, as set forth in Sambrook, et al. (1989) supra. For instance, mRNA can be isolated using various lytic enzymes or chemical solutions according to the procedures set forth in Sambrook, et al. (1989), supra or extracted by nucleic-acid-binding resins following the accompanying instructions provided by manufactures.

A preferred amplification method is PCR. PCR conditions used for each reaction are empirically determined. A number of parameters influence the success of a reaction. Among them are annealing temperature and time, extension time, Mg²⁺ ATP concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination.

The invention further provides the isolated polynucleotide operatively linked to a promoter of RNA transcription, as well as other regulatory sequences for replication and/or transient or stable expression of the DNA or RNA. As used herein, the term “operatively linked” means positioned in such a manner that the promoter will direct transcription of RNA off the DNA molecule. Examples of such promoters are SP6, T4 and T7. In certain embodiments, cell-specific promoters are used for cell-specific expression of the inserted polynucleotide. Vectors which contain a promoter or a promoter/enhancer, with termination codons and selectable marker sequences, as well as a cloning site into which an inserted piece of DNA can be operatively linked to that promoter are well known in the art and commercially available. For general methodology and cloning strategies, see GENE EXPRESSION TECHNOLOGY (Goeddel ed., Academic Press, Inc. (1991)) and references cited therein and VECTORS: ESSENTIAL DATA SERIES (Gacesa and Ramji, eds., John Wiley & Sons, N.Y. (1994)), which contains maps, functional properties, commercial suppliers and a reference to GenEMBL accession numbers for various suitable vectors. Preferably, these vectors are capable of transcribing RNA in vitro or in vivo.

Polynucleotides Of the Invention in a Host Cell

In a preferred embodiment of the present invention the recombinant system of polypeptide encoding polynucleotides is inserted into a host cell to provide a method to replicate the polynucleotide and a method to produce recombinant protein. The selection of a particular host cell will effect the level and quality of epitope loaded antigen presenting multimer produced and certain features of the host cell will be adjusted to optimize the production system. Thus, in a preferred embodiment of the present invention the host cell will be a cell that expresses β2 microglobulin, when the antigen presenting immunoglobulin fold domain is derived from an MHC class I molecule, in order to facilitate epitope binding and presentation. In a further embodiment of the invention the host cell will be selected to lack expression of a second antigen presenting molecule that binds the epitope encoded by the recombinant system of the invention. This, and other features of the host cell and recombinant system can be adjusted to optimize the production of epitope loaded antigen presenting multimer. Such features include, but are not limited to proteosome accessory proteins, proteosome beta-subunit components, TAP (transporter associated with antigen processing) protein, ER targeting sequences for the epitope peptide, and other factors involved in antigen processing, transport and binding to an antigen presenting molecule.

Methods of the Invention for Production of Fusion Polypeptides

The present invention also provides methods for producing antigen binding fusion polypeptides loaded with peptide epitopes and formed into multimers. In one aspect of the invention, fusion polypeptides are produced by introducing the recombinant system described above into a host cell and co-expressing the fusion polypeptide together with the corresponding T cell epitope in such a host cell. Methods for producing such recombinant proteins in host cells are well known in the art and suitable expression vectors for such purposes are described in detail above. As described above, the host cell should be selected to provide for optimal production of the polypeptide components of the invention and also to facilitate processing of the T cell epitope peptide as well as specific binding of this peptide to the fusion polypeptide. Furthermore, the levels of expression of the fusion polypeptide and T cell epitope peptide can be modulated to optimize the loading of peptide epitope into the antigen presenting domain of the fusion protein. For this purpose it is desirable to express the peptide epitope at relatively high levels to ensure that the fusion proteins are efficiently loaded with T cell epitope. This can be accomplished, for example, by selecting alternative promoter elements to direct expression of the two polynucleotides encoding these polypeptides. Promoters are chosen such that the peptide epitope encoding polynucleotide is transcribed at a relatively high level compared with the fusion polypeptide encoding polynucleotide. Various alternative methods for modulating the expression of the polynucleotides of the invention include, but are not limited to, adjusting the relative copy numbers of the polynucleotides, modulating the efficiency of translation of the polynucleotides, and altering the relative stabilities of mRNA's encoding the polypeptides.

In addition to producing fusion polypeptide loaded with T cell epitope peptide, the production method also provides for spontaneously forming polypeptide multimers by adjusting the production and purification conditions to allow efficient association of the oligomerization domains. The precise conditions required for multimer formation will depend on the specific oligomerization domain selected. For example, dimer formation of recombinant fusion proteins with leucine zipper domains has previously been demonstrated using fusions of a leucine zipper domain and green fluorescent protein (Katz et al. (1998) BioTechniques 25(2):298-302). In this example, the fusion protein multimers were formed spontaneously inside the host cell. Alternatively, dimer formation mediated by leucine zipper domains can be performed in vitro under appropriate conditions of protein concentration and buffer composition.

In many cases, it is desirable to produce the polypeptides of the invention by expressing polynucleotides that encode polypeptides with secretion signals on their amino termini. Thus the polypeptides can bind peptide antigen within the host cell and then be secreted into the extracellular space. Such secretion can facilitate purification of the resulting fusion polypeptides.

Where the oligomerization domain is a substrate binding domain or a peptide mimetic of biotin, multimer formation can be performed by addition of a suitable platform molecule, for example streptavidin, when the oligomerization domain is a peptide mimetic of biotin. Polypeptides secreted into the host cell culture medium can be rapidly purified by passage of the cell supernatant through a column of streptavidin immobilized on beads followed by elution with biotin to yield purified peptide epitope loaded monomers. Following removal of the biotin by dialysis, monomers can be mixed with streptavidin platform molecule to produce multimers.

Alternative methods for purification of the peptide antigen loaded fusion protein complex can also be used. For example, various separation techniques are well known in the art and can be employed to isolate purified fusion protein-epitope complexes. Such techniques include, but are not limited to gel filtration, ion exchange chromotography, reverse phase chromatography and hydrophobic interaction chromatography. Furthermore, many specific methods can be effective for purifying particular fusion proteins. For example, when a monoclonal antibody is available that recognizes the fusion protein, antibody affinity chromatography can be used. Alternatively, binding of the fusion protein to specific substrates or precipitation and enrichment by addition of protein specific reagents such as by immuno-precipitation can be used.

It will be clear to skilled practitioners of the art that further specific methods will be employed to produce and purify recombinant fusion polypeptides loaded with peptide antigen, depending on the specific polypeptides selected for the fusion molecule. Many effective purification procedures have been demonstrated for the polypeptides envisioned in this invention as oligomerization domains and it is precisely the variety and versatility of such polypeptide domains which has been designed to provide the present invention with a broad spectrum of applications and aspects.

A Recombinant System Comprising Polynucleotides of the Invention

The present invention also provides a recombinant system comprising a polynucleotide encoding an antigen presenting fusion protein and a second polynucleotide that encodes a T cell epitope which binds specifically to the antigen presenting domain of the fusion protein. While in natural antigen presentation systems, antigen processing and antigen presentation molecule synthesis are closely coordinated, prior methods for forming soluble antigen presenting multimers required a separate in vitro reaction to add and bind peptide antigen to purified antigen presenting molecules. The present invention provides a significant improvement on this process by combining the peptide epitope and antigen presenting fusion protein together in a coordinated recombinant system.

In a preferred embodiment of the invention, the antigen presenting fusion protein encoding polynucleotides are formatted adjacent to a promoter or promoter elements in an expression vector as described above. The polynucleotide molecules may be present on the same or different expression vector molecules. The key is to express both genes at the same time.

Analysis of peptide epitope-antigen binding has shown that the efficiency of binding of peptides to an antigen presenting molecule depends on the specific sequences of the epitope and corresponding antigen presenting molecule. Significant attention has been paid to identifying detailed features of the peptide epitope and of the antigen processing system that determine the efficiency of epitope binding to an antigen presenting molecule. Those skilled in the art will recognize a variety of specific features of the recombinant system which can be adjusted and optimized in order to achieve effective production of epitope loaded antigen presenting multimers. Such features include but are not limited to the specific amino acid sequence of the peptide epitope encoding polynucleotide; the specific nucleotide sequences of the expression vectors used to synthesize the polypeptides, especially features which determine the level of expression of polypeptide; the relative levels of expression of polypeptide epitope and antigen presenting fusion polypeptide; and the number of copies of the polypeptide encoding polynucleotide included in each expression construct.

The following examples are intended to illustrate how to make and assay the various compositions and methods of this invention.

APC:Target Cell Fusions

The present invention also provides methods for inducing in vivo an immune response against endogenous antigens by delivering an agent that genetically modifies a cell in a subject causing the cell to fuse with an antigen presenting dendritic cell. Fusion of such a target cell with a dendritic cell results in processing and presentation of endogenous antigens from the target cell. The antigens are then loaded by MHC molecules of the dendritic cell and presented to T cells in the context of these MHC molecules, thereby inducing an immune response against the target cells.

In one embodiment the agent comprises a molecule that stimulates the inherent ability of dendritic cells to engulf other cells. For example, the agent can deliver to a target cell a cell surface molecule that binds with high affinity to the surface of a dendritic cell, inducing the dendritic cell to engulf the target cell. Specific molecules with this property include, but at not limited to: 1) the Fc portion of an antibody molecule; 2) a protein or peptide covalently linked to accessible mannose residues; 3) the molecule phosphatidylserine; and 4) a molecule such as vitronectin and thrombospondin that binds to an □ᵥ□₃ integrin molecule that is present on the surface of a dendritic cell.

Such molecules can be delivered directly to the target cell, for example by direct injection of an effective amount of the agent at the site of the target cell. The agents can be further directed to the target cell by formulation in a suitable composition that facilitates introduction of the agent to the target cell. A variety of alternative in vivo delivery technique have been developed that will be known to those skilled in the art. Examples include, but are not limited to, compositions with various forms of liposomes, targeting by attachment to cell type specific antibody molecules, delivery with viral particles, and attachment to polypeptide ligand for specific cell surface receptors on the target cell.

In an alternative embodiment, the target cell are genetically modified to express on their surface a polypeptide molecule that simulates fusion with a dendritic cell. For example, the agent can comprise a gene delivery vehicle such as a viral vector or a transposon insertion vector that can insert into the target cell chromosome and turn on expression of the poplypeptide of interest. Methods of creating site specific insertions into mammalian genomes have been demonstrated and are known to those skilled in the art.

In one embodiment of the invention the agent comprises a polynucleotide that encodes a polypeptide wherein the polypeptide is a cell surface molecule that stimulates dendritic cells to engulf the target cell. The polynucleotide can be inserted into a gene delivery vehicle in which it is operatively linked to a promoter element that directs expression of the encoded polypeptide following introduction of the gene delivery vehicle into a target cell. Example of such gene delivery vehicles include viral and non-viral vectors as described above and methods for constructing and administering these vehicles are well known in the art.

When the agent comprises a polynucleotide an appropriate gene sequence can be isolated from a cDNA library using standard methods know to those of ordinary skill. For example, a gene specific probe can be generated using the PCR process with primers designed based on the known sequence of the gene of interest. When PCR is performed on an appropriate nucleic acid sample, for example genomic DNA or cDNA derived from a cell or tissue that expresses the gene of interest, a polynucleotide fragment comprising a portion of the gene of interest will be produced. This probe can then be detectably labeled in vitro and used to identify clones in a cDNA library that contain the gene sequence of interest. If only a portion of the gene encoding polynucloetide is obtained, it is possible to perform further techniques such as 5′ and 3′ RACE to obtain a complete coding sequence.

In a separate aspect of the invention the agent that induces fusion of the target cell with a dendritic cell comprises a viral envelope protein that exhibits tropism for dendritic cell surface proteins. For example, the agent can be the extracellular portion of the HIV-1 gp120 protein displayed on the surface of the target cell. The gp120 protein has affinity for CD4 and chemokine receptors on the surface of dendritic cells. Binding of a target cell surface bound HIV-1 gp120 molecule to a dendritic cell CD4 molecule will thus stimulate adhesion and fusion of a target cell and a dendritic cell.

In a separate aspect the viral envelope protein can be the measles virus envelope protein displayed on the surface of the target cell. Dendritic cells possess the receptor for the measles virus, thus providing a mechanism to facilitate binding and fusion of a target cell and a dendritic cell.

The present invention also provides a method where the agent comprises a polynucleotide encoding a viral envelope protein. Thus the agent can comprise such a polynucleotide inserted into a gene delivery vehicle and administered to a subject as described above.

In an alternative aspect of the invention the agent comprises a cell surface ligand for a receptor on the surface of a dendritic cell, a cell surface receptor for a ligand on the surface of a dendritic cell or a novel engineered protein that has affinity for a naturally occurring ligand or receptor on the surface of a dendritic cell.

In a particular embodiment, the agent comprises the ligand for receptors including, but not limited to CD11c, MHC class II molecules, MHC class I molecules, B7.1 and the mannose receptor. In another embodiment, the agent comprises a genetically engineered protein that has affinity for a naturally occurring ligand or receptor displayed on the surface of a dendritic cell. For example, the agent can be an antibody with specificity for the cell surface mannose receptor of dendritic cells engineered so that it is displayed on the surface of a target cell. Presentation of this molecule on the surface of the target cell will direct adherance of a dendritic cell and facilitate fusion of the cell pair,

The present invention also provides agents that genetically modify a target cell to express such genetically engineered proteins. Thus, the invention provides for polynucleotides comprising engineered nucleotide sequences that encode polypeptides with specific affinity for cell surface molecules on dendritic cells where the engineered polypeptides are designed to be presented on the surface of a target cell. Methods for constructing such engineered polynucleotides are well known to those practiced in the art. For example, appropriate polynucleotide sequences from cloned molecules can be precisely fused using standard recombinant DNA techniques and inserted into a gene delivery vehicle so that the specified engineered polypeptide can be expressed in a cell.

The invention further provides an agent that modifies both a target cell and a dendritic cell so that these cells present a complimentary pair of molecules with mutual binding affinity for each other such as a receptor ligand pair. For example the agent can comprise sreptavidin and a peptomimetic of biotin such as the peptide CHPQXC, where one of these molecules is displayed on the surface of target cell and he other is displayed on the surface of a dendritic cell.

The agent can further comprise polynucleotides that encodes these polypeptides inserted into delivery vehicles wherein the polynucleotides are operatively linked to suitable promoter elements. Thus, following administration of the agent the polypeptides can be expressed by the target cell and dendritic cell inducing adhesion and fusion of the target cell and dendritic cell.

Induction of cell fusion between a target cell and a dendritic cell using these methods will facilitate presentation of endogenous antigens derived from the target cell in the context of MHC molecules, providing an effective method to induce immune responses to these endogenous antigens.

Methods to Monitor Therapy

Monitoring of tumor regression in vivo or the use of assays to determine T cell response which are well known in the art can be utilized to determine if the object of the methods described herein have been achieved. For example, any method that will compare T cell number prior to and subsequent to therapy can be utilized. In addition, the induction of co-stimulatory molecules by these methods could also stimulate anergic or low affinity self-reactive CTL clones. Methods to assay for CTL clones include: standard ⁵¹Cr release assay as describe in Kawakami, et al. (1988) J. Exp. Med. 168:2183-91. Briefly, cytotoxic T cells are added to target cells previously loaded with ⁵¹Cr and one measures the release of ⁵¹Cr from the lysed target cells. Cytokine release assay as described in Kawakami, et al. (1994) PNAS 91:3515-19. Briefly, cytotoxic T cells are added to target cells and one measures the amount of IFNα released by ELISA. To measure the relative proportion of immune effector cells within a mixed population that recognize a particular target the Enzyme-linked immunospot (ELISPOT) assay is employed as described in Czerkinsky, et al. (1988) J. Immunol. Meths. 110:29-36. Briefly, 96 well nitrocellulose-bottomed plates are coated with an anti-cytokine antibody, generally anti-interferon-γ. Target cells and immune effector cells such as cytotoxic T cells (CTLs) are added to wells. Cytokine released from the CTLs is captured by the anti-interferon-α antibody and quantitated using a standard ELISA format.

Isolation, Culturing and Expansion of APCs including Dendritic cells

Various methods to isolate and characterize APCs including DCs have been known in the art. At least two methods have been used for the generation of human dendritic cells from hematopoietic precursor cells in peripheral blood or bone marrow. One approach utilizes the rare CD34⁺ precursor cells and stimulate them with GM-CSF plus TNF-α. The other method makes use of the more abundant CD34⁻ precursor population, such as adherent peripheral blood monocytes, and stimulate them with GMCSF plus IL-4 (see, for example, Sallusto et al. (1994), supra).

In one aspect of the invention, the method described in Romani et al (1996), infra and Bender et al. (1996) J. Immunol. Methods 196:121-135, is used to generate both immature and mature dendritic cells from the peripheral blood mononuclear cells (PBMC) of a mammal, such as a murine, simian or human. Briefly, isolated PBMC are pre-treated to deplete T- and B-cells by means of an immunomagnetic technique. Lymphocyte-depleted PBMC are then cultured for 7 days in RPMI medium, supplemented with 1% autologous human plasma and GM-CSF/IL-4, to generate dendritic cells. Dendritic cells are non-adherent as opposed to their monocyte progenitor. Thus, on day 7, non-adherent cells are harvested for further processing.

The dendritic cells derived from PBMC in the presence of GM-CSF and IL-4 are immature, in that they can lose the nonadherence property and revert back to macrophage cell fate if the cytokine stimuli are removed from the culture. The dendritic cells in an immature state are very effective in processing native protein antigens for the MHC class II restricted pathway (Romani et al. (1989) J. Exp. Med. 169:1169).

Further maturation of cultured dendritic cells is accomplished by culturing for 3 days in a macrophage-conditioned medium (CM), which contains the necessary maturation factors. Mature dendritic cells are less able to capture new proteins for presentation but are much better at stimulating resting T cells (both CD4⁺ and CD8⁺) to grow and differentiate.

Mature dendritic cells can be identified by their change in morphology, such as the formation of more motile cytoplasmic processes; by their nonadherence; by the presence of at least one of the following markers: CD83, CD68, HLA-DR or CD86; or by the loss of Fc receptors such as CD115 (reviewed in Steinman (1991) Annu. Rev. Immunol. 9:271.)

Alternatively, others have reported that a method for upregulating (activating) dendritic cells and converting monocytes to an activated dendritic cell phenotype. This method involves the addition of calcium ionophore to the culture media convert monocytes into activated dendritic cells. Adding the calcium ionophore A23187, for example, at the beginning of a 24-48 hour culture period resulted in uniform activation and dendritic cell phenotypic conversion of the pooled “monocyte plus DC” fractions: characteristically, the activated population becomes uniformly CD14 (Leu M3) negative, and upregulates HLA-DR, HLA-DQ, ICAM-1, B7.1, and B7.2. Furthermore this activated bulk population functions as well on a small numbers basis as a further purified.

Specific combination(s) of cytokines have been used successfully to amplify (or partially substitute) for the activation/conversion achieved with calcium ionophore: these cytokines include but are not limited to G-CSF, GM-CSF, IL-2, and IL-4. Each cytokine when given alone is inadequate for optimal upregulation.

In one embodiment, the APCs and cells expressing one or more antigens are autologous. In another embodiment, the APCs and cells expressing the antigen are allogeneic, i.e., derived from a different subject.

Presentation of Antigen by the APC

In certain aspects of the invention, APC loaded with antigen are administered to the subject. Peptide fragments from antigens must first be bound to peptide binding receptors (MHC class I and class II molecules) that display the antigenic peptides on the surface of the APCs. Palmer E. and Cresswell (1998) Annu. Rev. Immunol. 16:323 and Germain R. N. (1996) Immunol. Rev. 151:5. T lymphocytes produce an antigen receptor that they use to monitor the surface of APCs for the presence of foreign peptides. The antigen receptors on CD4⁺ T cells recognize antigenic peptides bound to MHC class II molecules whereas the receptors on CD8⁺ T cells react with antigens displayed on class I molecules. For a general review of the methods for presentation of exogeneous antigen by APC, see Raychaudhuri and Rock (1998) Nature Biotechnology 16:1025.

For purposes of immunization, antigens can be delivered to antigen-presenting cells as protein/peptide or in the form of polynucleotides encoding the protein/peptide ex vivo or in vivo. The methods described below focus primarily on DCs which are the most potent, preferred APCs.

Several different techniques have been described to produce genetically modified APCs. These include: (1) the introduction into the APCs of polynucleotides that express antigen or fragments thereof; (2) infection of APCs with recombinant vectors to induce endogenous expression of antigen; and (3) introduction of tumor antigen into the DC cytosol using liposomes. (See, Boczkowski D. et al. (1996) J. Exp. Med. 184:465; Rouse et al. (1994) J. Virol. 68:5685; and Nair et al. (1992) J. Exp. Med. 175:609). For the purpose of this invention, any method that allows for the introduction and expression of the heterologous or non-self antigen and presentation by the MHC on the surface of the APC is within the scope of this invention.

Several techniques have been described for the presentation of exogenous protein and/or peptide by the APC. These techniques are briefly described below.

Antigen Pulsing

Pulsing is accomplished in vitro/ex vivo by exposing APCs to antigenic protein or peptide(s). The protein or peptide(s) are added to APCs at a concentration of 1-10 μm for approximately 3 hours. Paglia et al. (1996) J. Exp. Med. 183:317, has shown that APC incubated with whole protein in vitro were recognized by MHC class I-restricted CTLs, and that immunization of animals with these APCs led to the development of antigen-specific CTLs in vivo.

Antigen Painting

Another method which can be used is termed “painting”. It has been demonstrated that glycosyl-phosphotidylinositol (GPI)-modified proteins possess the ability to reincorporate themselves back into cell membranes after purification. Hirose et al. (1995) Methods Enzymol. 250:582; Medof et al. (1984) J. Exp. Med. 160:1558; Medof (1996) FASEB J. 10:574; and Huang et al. (1994) Immunity 1:607, have exploited this property in order to create APCs of specific composition for the presentation of antigen to CTLs. Expression vectors for β2-microglobulin and the HLA-A2.1 allele were first devised. The proteins were expressed in Schneider S2 Drosophila melanogaster cells, known to support GPI-modification. After purification, the proteins could be incubated together with a purified antigenic peptide which resulted in a trimolecular complex capable of efficiently inserting itself into the membranes of autologous cells. In essence, these protein mixtures were used to “paint” the APC surface, conferring the ability to stimulate a CTL clone that was specific for the antigenic peptide. Cell coating was shown to occur rapidly and to be protein concentration dependent. This method of generating APCs bypasses the need for gene transfer into the APC and permits control of antigenic peptide densities at the cell surfaces.

Foster APC

Foster APCs are derived from the human cell line 174xCEM.T2, referred to as T2, which contains a mutation in its antigen processing pathway that restricts the association of endogenous peptides with cell surface MHC class I molecules (Zweerink et al. (1993) J. Immunol. 150:1763). This is due to a large homozygous deletion in the MHC class II region encompassing the genes TAP1, TAP2, LMP1, and LMP2, which are required for antigen presentation to MHC class I-restricted CD8⁺ CTLs. In effect, only “empty” MHC class I molecules are presented on the surface of these cells. Exogenous peptide added to the culture medium binds to these MHC molecules provided that the peptide contains the allele-specific binding motif. These T2 cells are referred to herein as “foster” APCs. They can be used in conjunction with this invention to present the heterologous, altered or control antigen.

Transduction of T2 cells with specific recombinant MHC alleles allows for redirection of the MHC restriction profile. Libraries tailored to the recombinant allele will be preferentially presented by them because the anchor residues will prevent efficient binding to the endogenous allele.

High level expression of MHC molecules makes the APC more visible to the CTLs. Expressing the MHC allele of interest in T2 cells using a powerful transcriptional promoter (e.g., the CMV promoter) results in a more reactive APC (most likely due to a higher concentration of reactive MHC-peptide complexes on the cell surface).

Alternatively, it has been shown that fused cells lack functional hypoxanthine-guaninephosphoribosyl transferase (“HGPRT”) enzyme and are, therefore, resistant to treatment with the compound hypoxanthine aminopterin thymidine (“HAT”). Accordingly, to select these cells HAT can be added to the culture media. However, unlike conventional HAT selection, hybrid cell cultures should not be exposed to the compound for more than 12 days. The APCs as described above can be assayed for antigen(s) expression as described below.

Assaying Antigen-Specificity

The cells described herein are selected for their ability to actively lyse the cells expressing the specific antigen. Cytolytic activity of the cells can be measured in various ways, including, but not limited to, tritiated thymidine incorporation (indicative of DNA synthesis), and examination of the population for growth or proliferation, e.g., by identification of colonies. (See, e.g., WO 94/21287). In another embodiment, the tetrazolium salt MTT (3-(4,5-dimethyl-thazol-2-yl)-2,5-diphenyl tetrazolium bromide) may be added (Mossman (1983) J. Immunol. Methods 65:55-63; Niks and Otto (1990) J. Immunol. Methods 130:140-151). Succinate dehydrogenase, found in mitochondria of viable cells, converts the MTT to formazan blue. Thus, concentrated blue color would indicate metabolically active cells. Similarly, protein synthesis may be shown by incorporation of ³⁵S-methionine. In still another embodiment, cytotoxicity and cell killing assays, such as the classical chromium release assay, may be employed to evaluate epitope-specific CTL activation. Other suitable assays will be known to those of skill in the art.

In vivo, Ex vivo or In vitro Introduction of Gene Delivery Vehicles

The gene delivery vehicles carrying the polynucleotides described herein can be introduced in the host cell in vivo, ex vivo or in vitro. In one embodiment, the gene delivery vehicles are introduced in vivo to a subject. The vehicle may be introduced by transdermal, oral, subcutaneous, intravenous, intramuscular or parenteral modes (Wan et al. (1997) Human Gene Therapy 8:1355-63). For example, where the tumor to be treated includes a site such as a skin lesion or nodule (e.g., melanoma), the gene delivery vehicle(s) could be applied topically to the skin or injected subcutaneously into the nodule or lesion. Liposomal gene delivery vehicles, for example, can penetrate into the epidermis when topically applied.

In another embodiment, ex vivo gene therapy techniques can be employed. Here, the molecules of gene delivery vehicles carrying the genes encoding the molecules are introduced into cells outside of the subject and the transduced cells reintroduced into the subject. Preferably, the cells are obtained from a biopsy sample taken from the subject. The cells can be cultured and a gene delivery vehicle carrying a polynucleotide encoding an APBP introduced into the cells in culture. Optionally, transduction with a cytotoxic agent can also be performed in culture. The cells can then be reintroduced into the subject and the cytotoxic agent activated in vivo, causing release of the APBP:peptide complexes in the subject.

In yet another embodiment, the method described herein can be practiced in vitro. For example, a tumor biopsy sample can be isolated as described below. In culture, molecules or polynucleotides encoding the APBP, cytotoxic agent and, optionally, APC recruitment factor can then be introduced using one or more gene delivery vehicles. In addition, APCs and naive immune effector cells should be added—the APCs will present the tumor-specific antigens and educate the naive immune cells. The antigen-specific immune effector cells can then be reintroduced in the subject to specifically recognize and destroy the tumor cells.

Assessing Efficacy of Gene Transfer In vitro or In vivo

The efficacy of gene transfer into the cells of the subject can be monitored by any method known in the art. For example, as described above, a reporter or marker gene can be included in the gene delivery vehicle to facilitate identification of those cells into which the vehicle is successfully incorporated (Kass-Eisler, et al. (1994) Gene Therapy 1:395-402). Especially in the in vitro and ex vivo contexts, marker genes may prove especially helpful. Screening markers or reporter genes are genes that encode a product that can readily be assayed. Non-limiting examples of screening markers include genes encoding for green fluorescent protein (GFP) or genes encoding for a modified fluorescent protein.

Preferably, the marker gene included in the delivery vehicle is a selectable marker. A “positive” selectable marker gene encodes a product that enables only the cells that carry the gene to survive and/or grow under certain conditions. For example, plant and animal cells that express the introduced neomycin resistance (Neor) gene are resistant to the compound G418. Cells that do not carry the Neor gene marker are killed by G418. Negative selectable marker genes encode a product that allows cells expressing that product to be selectively killed. For example, as described above the conditionally activated cytotoxic agent may also be a selectable marker such as HSV-tk. Cells expressing this gene can be selectively killed using gancyclovir or acyclovir.

Other methods that can be employed to determine the extent of gene transfer include:

(1) quantitation of vector specific DNA by PCR;

(2) quantitation of transgene specific mRNA by RT-PCR;

(3) quantitation of the amount of transgene product in the serum by ELISA if the transgene encodes a soluble, secreted factor such as (α-1-anti-trypsin;

(4) quantitation of the amount of anti-transgene product antibody in the serum by ELISA; and

(5) quantitation of the amount of transgene product biological activity within the target tissue by homogenization of the tissue and assaying for enzymatic activity or treating the tissue with a suitable substrate that will be converted by the transgene product into a compount that can be quantitated by calorimetric methods.

The following examples are intended to illustrate, and not limit the invention.

### EXAMPLE 1

### Melanoma or Head and Neck Therapy with Cytotoxic Agent Lysis

Patients with accessible lesions of melanoma or head and neck cancer patients are injected with a vector or vectors encoding the antigenic peptide binding protein, the cytotoxic factor and an APC recruitment factor. If the cytotoxic factor is HSV thymidine kinase, the prodrug gancyclovir would be administered systemically via IV infusion. Tumor regression and anti-tumor cell immune responses are monitored.

### EXAMPLE 2

### gp100 Target for Exogenous Antigen Induced Lysis

gp100 is a tumor antigen associated with melanoma but it is not expressed by breast cancer cells. Dendritic cells genetically modified to express the human gp100 antigen are injected into a breast cancer patient to provoke an anti-gp100 T cell response. When there is a measurable gp100 specific response, an adenoviral vector encoding gp100 is injected into the breast tumor to force cancer cells to express the gp100 antigen. Given the patient has been primed with the gp100 vaccine, gp100 specific cytotoxic T cells should recognize and lyse the breast cancer cells that have been genetically modified to express gp100 . Endogenous antigens released from lysed breast cancer cells are captured by APCs leading to the generation of a secondary immune response targeted at all the breast cancer cells whether they have been genetically modified to express gp100 or not.

### EXAMPLE 3

### E. coli □-galactosidase Target for Exogenous Antigen Induced Lysis

E. coli β-galactosidase is a potent foreign antigen that can provoke cellular immune responses in both patients as well as experimental animals. Dendritic cells genetically modified to express E. coli β-galactosidase are injected into a cancer patient to provoke an anti-E. coli β-galactosidase T cell response. When there is a measurable E. coli α-galactosidase specific response, an adenoviral vector encoding E. coli β-galactosidase is injected into the tumor to force cancer cells to express the E. coli β-galactosidase protein. Given the patient has been primed with the E. coli β-galactosidase “vaccine.” E. coli β-galactosidase specific cytotoxic T cells should recognize and lyse the cancer cells that have been genetically modified to express E. coli β-galactosidase. Endogenous antigens released from lysed cancer cells are captured by APCs leading to the generation of a secondary immune response targeted at all cancer cells whether they have been genetically modified to express E. coli β-galactosidase or not.

### EXAMPLE 4

### Exploiting the Inherent Ability of Scavenger Cells (DCs, Macrophages) to Engulf Microbes and Cells

In one aspect, tumor cells are transduced with a gene that will cause the Fc portion of an antibody to be displayed on the surface of the tumor cells. Scavenger cells recognize the “marked” tumor cells via the Fc receptor.

In a separate aspect, tumor cells are transduced with a gene that will cause a protein or peptide with accessible mannose residues to be displayed on the surface of the tumor cells. Scavenger cells recognize the “marked” tumor cells via the mannose receptor.

In a further aspect, tumor cells are transduced with a gene that will cause phosphatidylserine to be displayed on the surface of the tumor cells. Scavenger cells recognize the “marked” tumor cells by physphatidylserine receptors.

Tumor cells are transduced with a gene that will cause vitronectin or thrombospondin to be displayed on the surface of the tumor cells. Scavenger cells recognize the “marked” tumor cells via αVβ3/CD47/CD36 interactions.

### EXAMPLE 5

### Exploiting the Tropism of Viral Envelope Proteins for DC Cell Surface Proteins

Tumor cells are transduced with a gene that will cause the extracellular portion of the HIV-1 gp120 envelope protein to be displayed on the surface of the tumor cells. The gp120 protein has affinity for CD4 and chemokine receptors on the surface of DCs thus union of transduced tumor cells with DCs is favored when the cells encounter one another.

Tumor cells are transduced with a gene that will cause the measles virus envelope protein to be displayed on the surface of the tumor cells. DCs possess the receptor for the measles virus thus union of transduced tumor cells with DCs is favored when the cells encounter one another.

Labeling the cell surface of tumor cells with a binding protein or ligand that has affinity for a cell surface ligand or receptor naturally displayed on the surface of dendritic cells.

### EXAMPLE 6

### Modification of Both Tumor Cells and APCs to Introduce Receptor:Ligand Pairs

Tumor cells are transduced with a gene that will cause streptavidin to be displayed on the cell surface. Biotinylated DCs or DCs transduced with a gene that will cause a peptomimetic of biotin (such as CHPQXC) to be displayed on the cell surface are administered to the host. Interaction of the streptavidin displaying tumor cells with biotin or biotin mimetic labels DCs favors the union of tumor cells with DCs.

Tumor cells are transduced with a gene that will cause an antibody to be displayed on the cell surface. DCs transduced with a gene that will cause the cognate ligand for the antibody to be displayed on the cell surface are administered to the host. Interaction of the antibody tagged tumor cells with ligand tagged DCs favors the union of tumor cells with DCs.

Although the foregoing invention has been described in some detail by way of illustration and example for purposes of clarity of understanding, it will be apparent to those skilled in the art that certain changes and modifications will be practiced. Therefore, the description and examples should not be construed as limiting the scope of the invention, which is delineated by the appended claims.

What is claimed is:

1. A method for inducing an immune response to an endogenous antigen in a subject comprising delivering a cytotoxic agent,
wherein said cytotoxic agent is an effective amount of one or more of herpes simplex virus thymidine kinase (HSV-tk), gancicolvir, or a rejection antigen, that stimulates in vivo loading of an endogenous antigen,
wherein said endogenous antigen is a tumor associated antigen or a viral antigen, into an antigenic binding protein (APBP) molecule,
wherein said APBP molecule is selected the group consisting of a heat shock protein (HSP), a soluble major histocompatibility complex (MHC) class I molecule, an antigen presenting matrix, a multimer of soluble MHC class I molecules and an antibody engineered to bind antigen peptides, under conditions so that the endogenous antigen is presented to a T cell and the agent induces lysis of said target cell.

2. A method for inducing lysis of a target cell in a subject, comprising the steps of:
a. inducing an immune response to an exogenous rejection antigen in the subject, comprising (i) delivering to the subject an effective amount of a composition comprising the exogenous rejection antigen that presents the exogenous rejection antigen on the cell surface, or (ii) delivering to the subject an effective amount of an immune effector cell population educated with the exogenous rejection antigen; and
b. delivering to a target cell, wherein the target cell is a tumor cell or a virally infected cell, in the subject an effective amount of a polynucleotide encoding the exogenous antigen, thereby inducing lysis of the target cell in the subject.

3. The method of claim 2 further comprising delivering an effective amount of an antigen presenting cell recruitment factor.

4. A fusion polypeptide comprising a T cell antigen presenting domain fused to an oligomerization domain.

5. The fusion polypeptide of claim 4, wherein the T cell antigen presenting domain comprises a plurality of immunoglobulin fold domains of an MHC class I molecule.

6. The fusion polypeptide of claim 4 wherein the oligomerization domain is selected from the group consisting of a peptide mimetic of a ligand, and a self-assembling protein.

7. An isolated polynucleotide comprising a nucleic acid sequence encoding the fusion polypeptide of claim 4.

8. A gene delivery vehicle comprising the polynucleotide of claim 7.

9. A host cell comprising the polynucleotide of claim 7.

10. A host cell comprising the polypeptide of claim 4.

11. A recombinant system comprising the isolated polynucleotide of claim 7 and a second polynucleotide that encodes a T cell epitope which binds specifically to the antigen presenting domain of the fusion polypeptide.

12. A method of producing an antigen presenting multimer comprising expressing a recombinant system comprising the isolated polynucleotide of claim 7 and a second polynucleotide that encodes a T cell epitope, said T cell epitope selected from the group consisting of a tumor cell antigen, a pathogenic antigen, and a self-antigen, which binds specifically to the antigen presenting domain of the fusion polypeptide, under conditions which allow the formation of antigen presenting multimers and isolating the multimer.

13. A method of detecting an antigen specific T cell comprising contacting peripheral blood lymphocytes with antigen presenting multimers of claim 12 under conditions which allow antigen specific binding to T cells, and detecting the multimer-T cell complex.

14. A method of isolating an antigen specific T cell comprising purifiying the antigen specific T cells of claim 13.

15. The T cell isolated by the method of claim 14.

16. A method of expanding a population of antigen specific T cells comprising culturing the cell of claim 15.

17. The T cell population expanded by the method of claim 16.

18. A method of enhancing an immune response in a subject comprising administering to the subject an expanded population of antigen specific T cells of claim 17.
